(19) United States
(12) Patent Application Publication
(10) Pub. No.: US 2023/0165996 A1
(43) Pub. Date: Jun. 1, 2023

(54) APPARATUS, METHOD, AND SYSTEM FOR THE STERILIZATION AND PURIFICATION OF AN INDOOR ENVIRONMENT

(71) Applicant: PURTEC, INC., Las Vegas, NV (US)

(72) Inventor: Samuel R Edwards, JR., Las Vegas, NV (US)

(21) Appl. No.: 17/921,916

(22) PCT Filed: Apr. 27, 2021
(86) PCT No.: PCT/US2021/029312

§ 371 Date: Oct. 27, 2022

Related U.S. Application Data
(60) Provisional application No. 63/101,245, filed on Apr. 27, 2020.

(51) Int. Cl. A61L 9/20

(52) U.S. Cl. CPC A61L 9/20; A61L 2202/14; A61L 2202/25

(57) ABSTRACT

An apparatus, method, and system for the sterilization and purification of an indoor environment are disclosed. In an example, an apparatus includes a processor, at least one ultraviolet lighting emitting diode (“LED”), an active oxygen generator, and a proximity sensor. The processor is configured to activate at least one of the active oxygen generator or the at least one ultraviolet LED to provide air/surface purification and/or sterilization at a designated time, a designated condition, or upon receiving an instruction. The processor is also configured to receive a signal from the proximity sensor indicative of a presence individual. In response, the processor pauses activation of at least one of the active oxygen generator or the at least one ultraviolet LED, and resumes activation of at least one of the active oxygen generator or the at least one ultraviolet LED when the presence of the individual is no longer detected.

### BACKGROUND

The present disclosure relates generally to indoor sterilization and purification, and, in particular, to using contamination monitoring for determining which sterilization and/or purification modalities to activate.

### BACKGROUND

As of 2021, the indoor air/surface purification and sterilization market is estimated to be around $2 billion. With the recent onset of the SARS-CoV-2 virus and the COVID-19 disease it causes, it is estimated that this market could at least double within the next few years as individuals attempt to cleanse their homes and offices. Further, as society places as emphasis on maintaining sterile environments to reduce disease spread (including common flus), the increased demand is expected to continue even as vaccines minimize the impact of the SARS-CoV-2 virus.

Known air purification devices typically use one or more filters to remove contaminates for cleansing air. An issue with filters is that they have to be frequently replaced. In some instances, air filters have be changed every few weeks. However, many individuals neglect to change the air filters either due to forgetfulness or to save money. As a result, the air filters become full over time, thereby reducing the effectiveness of the air purification device. While some manufacturers have attempted to overcome this issue by designing washable filters, many individuals neglect to even wash these filters.

Another known issue with air and surface purification devices is limited control. Many known devices only have settings for on/off and a fan speed. These controls are often manual or controlled via a timer based on an individual's discretion. However, most individuals are not aware of the exact level of contamination within a given indoor environment to be able to accurately gauge how long and at what intensity a device is to be activated. This results in some individuals powering their devices infrequently, which fails to eliminate contaminants. Alternatively, some individuals leave their devices on virtually all the time, which is inefficient. Further, some air purification devices emit activated oxygen (i.e., ozone), which can become an irritant at high concentrations when a device is powered for an extended duration.

### SUMMARY

The apparatus, method, and system of the present disclosure relate to air/surface purification and/or sterilization using contamination monitoring. The apparatus, method, and system are configured to provide air/surface purification and/or sterilization using one or more ultraviolet (“UV”) lighting emitting diodes (“LEDs”), such as UV-A and/or UV-C LEDs. The apparatus, method, and system are also configured to provide air/surface purification and/or sterilization using an active oxygen generator and/or ultrasonic speakers. The different purification and sterilization modalities enable the apparatus, method, and system to optimize decontamination based on detected environmental conditions and/or containments.

To detect indoor environmental conditions and/or containments, the apparatus, method, and system includes a temperature sensor, a humidity sensor, a barometric pressure sensor, a formaldehyde sensor, one or more air component sensors, and/or one or more volatile organic compound (“VOC”) sensors. The air component sensors may include one or more sensors to provide for the detection of combustible gas/smoke, alcohol vapors, methane, propane, butane, liquefied petroleum, liquid natural gas, carbon monoxide, hydrogen, ozone, ammonia sulfide, and/or benzene vapor. The sensors enable the apparatus, method, and system to provide the correct air/surface purification and/or sterilization modality for a sufficient duration to optimize decontamination of an indoor area.

In some embodiments, the apparatus, method, and system are configured to provide air/surface purification and/or sterilization at one or more scheduled times/days. Further, the apparatus, method, and system are configured to provide air/surface purification and/or sterilization upon detection of containments. The apparatus, method, and system are configured to optimize the sterilization and/or purification modality activated based on environmental conditions. For example, the use of UV-C LEDs is less effective when humidity levels are over 70%. However, ozone oxidation is optimized when humidity levels are over 70%. After detecting that humidity levels are greater than 70%, the apparatus, method, and system are configured to select ozone purification and/or sterilization rather than UC-C LED purification and/or sterilization.

Generally, the use of UV-A and/or UV-C LEDs, active oxygen, and/or ultrasonic waves may be mildly irritating to a user. To ensure users are not present when air/surface purification and/or sterilization, the example apparatus, method, and system are configured to include one or more proximity sensors that provide space configuration and room occupancy information. Upon detection of a user, the apparatus, method, and system are configured to pause any active sterilization and/or purification modalities. Further, the apparatus, method, and system are configured to delay the start of any scheduled sterilization and/or purification modalities until a certain time duration (e.g., one minute, two minutes, ten minutes, etc.) after which a user departed a monitored indoor area. In some instances, apparatus, method, and system use detection of one or more individuals within a monitored space to trigger one or more sterilization and/or purification modalities after their detected departure.

In some embodiments, apparatus, method, and system may use one sterilization and/or purification modality to negate another sterilization and/or purification modality from irritating an individual. For example, the apparatus, method, and system may generate ozone for a specified duration for sterilization and/or purification. After the ozone generation, the apparatus, method, and system detects that an ozone level is above a defined threshold (e.g., greater than 20 or 30 parts per billion (“ppb”)). The apparatus, method, and system are configured to activate an internal fan to provide air circulation to dissipate the ozone while activating one or more LEDs to stimulate ozone decay until the detected ozone level falls below the threshold. Such a configuration provides efficient air/surface purification and/or sterilization to an indoor environment when individuals are not present and ensures the indoor environment does not contain irritants when the individuals return.

An example system includes a beacon apparatus configured to detect containments and provide air/surface purification and/or sterilization for a relatively large area (e.g., 150 to 1500 square feet (“ft²”)). The example system may also include one or more hubs that are communicatively coupled to the beacon apparatus. Each hub is configured to provide air/surface purification and/or sterilization for a relatively small area (e.g., 10 to 150 ft²). The hubs are configured to provide air/surface purification and/or sterilization for areas that may not be reachable by the beacon apparatus (or within a shadow of the beacon apparatus). Depending on user configuration, an indoor environment may have as few as one beacon apparatus and zero hubs up to tens of beacon apparatus and hundreds of hubs, such as on a passenger vessel or plane, hotel, mall, museum, stadium, or conference center.

In some embodiments, the beacon apparatus and/or the hub include a wireless transceiver to enable communicatively coupling to at least one of a Wi-Fi network, a Zigbee® enabled device, and/or a Bluetooth® enabled device. The beacon apparatus and/or the hub configured are configured to transmit status information and/or air quality information to, for example, an application operating on a user device. The beacon apparatus and/or the hub are also configured to receive instructions from the application operating on the user device to begin one or more purification and/or sterilization modalities immediately, at a scheduled time, and/or at a detected condition.

The beacon apparatus and one or more nodes are configured to operate together to detect containments throughout an indoor area. The beacon may receive messages from the one or more nodes (in addition to its own detection) indicative of a detected air quality and/or indicative of a presence of an individual. The beacon apparatus may use the received messages to determine a total air quality and/or presence of one or more individuals within a monitored area. The beacon apparatus may be configured to determine which purification/sterilization modalities should be activated at each node and transmit a corresponding instruction to cause each of the nodes to operate accordingly. The beacon apparatus may also determine a duration the one or more purification/sterilization modalities are to be activated based, for example, on a level of contamination. In some embodiments, one or a few nodes may be activated for longer durations while other nodes may be inactive or activated for shorter durations. The beacon apparatus may also receive from a node information indicative of a pause due to detecting a presence of an individual. The beacon apparatus may use the presence information to pause other nodes in a same room or vicinity of the node that made the detection. The example beacon apparatus and nodes accordingly form a connected network of sensors and purification/sterilization modalities to more efficiently provide air/surface purification and/or sterilization.

In light of the disclosure herein and without limiting the disclosure in any way, in a first aspect of the present disclosure, which may be combined with any other aspect listed herein, a purification and sterilization apparatus includes a housing having a top side, a bottom side, a cylindrical face between the top side and the bottom side. The apparatus also includes at least one ultraviolet (“UV-C and/or UV-A”) lighting emitting diode (“LED”) supported by the housing, an active oxygen generator located within the housing, at least one proximity sensor located within the housing, and a processor located within the housing. The apparatus further includes a memory storing machine readable instructions, which when executed by the processor, cause the processor to activate at least one of the active oxygen generator or the at least one LED to provide air/surface purification and/or sterilization at a designated time, a designated condition, or upon receiving an instruction, receive a signal from the at least one proximity sensor indicative of a presence individual, pause activation of at least one of the active oxygen generator or the at least one LED, and resume activation of at least one of the active oxygen generator or the at least one LED when the presence of the individual is no longer detected for at least a time threshold.

In a second aspect of the present disclosure, which may be combined with any other aspect listed herein, the purification and sterilization apparatus further includes at least one air sensor located within the housing, and the memory stores additional machine readable instructions, which when executed by the processor, cause the processor to determine activation of the active oxygen generator is to be stopped based on the designated time or upon receiving a second instruction, determine from at least one signal from the air sensor that an ozone concentration is above an ozone threshold, and cause the at least one ultraviolet LED to activate to reduce the ozone concentration below the ozone threshold.

In a third aspect of the present disclosure, which may be combined with any other aspect listed herein, the ozone threshold is at least 20 parts per billion.

In a fourth aspect of the present disclosure, which may be combined with any other aspect listed herein, the at least one air sensor includes at least one of a formaldehyde sensor, one or more air component sensors, or one or more volatile organic compound (“VOC”) sensors, and the designated condition includes a detection by the processor of a containment above a threshold level using at least one signal from the at least one air sensor.

In a fifth aspect of the present disclosure, which may be combined with any other aspect listed herein, the one or more air component sensors are configured to provide for the detection of at least one of ozone, carbon dioxide, combustible gas/smoke, alcohol vapors, methane, propane, butane, liquefied petroleum, liquid natural gas, carbon monoxide, hydrogen, ozone, ammonia sulfide, or benzene vapor.

In a sixth aspect of the present disclosure, which may be combined with any other aspect listed herein, the at least one air sensor includes at least one of a temperature sensor, a humidity sensor, or a barometric pressure sensor.

In a seventh aspect of the present disclosure, which may be combined with any other aspect listed herein, the memory stores additional machine readable instructions, which when executed by the processor, cause the processor to determine a relative humidity of ambient air is greater than a humidity threshold, and activate the active oxygen generator while refraining from activing the at least one ultraviolet LED.

In an eighth aspect of the present disclosure, which may be combined with any other aspect listed herein, the memory stores additional machine readable instructions, which when executed by the processor, cause the processor to determine the relative humidity of ambient air is less than the humidity threshold, and activate the at least one ultraviolet LED while refraining from activing the active oxygen generator.

In a ninth aspect of the present disclosure, which may be combined with any other aspect listed herein, the humidity threshold is between 65% and 75% relative humidity.

In a tenth aspect of the present disclosure, which may be combined with any other aspect listed herein, the top side of the housing includes a cylindrical section, and a plurality of the ultraviolet LEDs are placed around a circumference of the cylindrical section.

In an eleventh aspect of the present disclosure, which may be combined with any other aspect listed herein, the apparatus further includes a motor, and an actuator arm connected to the motor and the cylindrical section, wherein the motor is configured to cause the actuator arm to raise and lower the cylindrical section with respect to the housing such that the plurality of the ultraviolet LEDs are exposed when the cylindrical section is in a raised position and hidden from view when the cylindrical section is in a retracted position.

In a twelfth aspect of the present disclosure, which may be combined with any other aspect listed herein, at least some of the plurality of the ultraviolet LEDs are configured to emit light in the 250 to 270 nanometer (“nm”) wavelength range and other of the at least some of the plurality of the ultraviolet LEDs are configured to emit light in the 390 to 420 nm wavelength range.

In a thirteenth aspect of the present disclosure, which may be combined with any other aspect listed herein, the cylindrical face includes a first vent adjacent to the top side, and a second vent adjacent to the bottom side.

In a fourteenth aspect of the present disclosure, which may be combined with any other aspect listed herein, the active oxygen generator includes an ozone ionizer plate, and a fan configured to pull ambient air through the second vent and cause ozone to be emitted through the first vent when the ozone ionizer plate is active.

In a fifteenth aspect of the present disclosure, which may be combined with any other aspect listed herein, the time threshold is between five seconds and fifteen minutes.

In a sixteenth aspect of the present disclosure, which may be combined with any other aspect listed herein, the apparatus further includes at least one ultrasonic speaker within the housing, the at least one ultrasonic speaker configured to emit a waveform having a frequency between 20 and 80 kHz, a sound pressure level between 80 and 150 dB, and an angle of radiation between 45° and 180°.

In a seventeenth aspect of the present disclosure, which may be combined with any other aspect listed herein, the apparatus further includes a display screen provided on the cylindrical face and including at least one of a touchscreen or input buttons, wherein the memory stores additional machine readable instructions, which when executed by the processor, cause the processor to receive the instruction via the display screen.

In an eighteenth aspect of the present disclosure, which may be combined with any other aspect listed herein, the memory stores additional machine readable instructions, which when executed by the processor, cause the processor to display a status or an air quality indication on the display screen.

In a nineteenth aspect of the present disclosure, which may be combined with any other aspect listed herein, the apparatus further includes a transceiver for communicatively coupling the processor to a user device, wherein the memory stores additional machine readable instructions, which when executed by the processor, cause the processor to receive the instruction via the transceiver from an application operating on the user device.

In a twentieth aspect of the present disclosure, which may be combined with any other aspect listed herein, the memory stores additional machine readable instructions, which when executed by the processor, cause the processor to transmit a status or an air quality indication for display by the application on the user device.

In a twenty-first aspect of the present disclosure, which may be combined with any other aspect listed herein, the apparatus further includes a transceiver for communicatively coupling the processor to another purification and sterilization apparatus or a hub configured as a smaller version of the purification and sterilization apparatus.

In a twenty-second aspect, any of the features, functionality, and alternatives described in connection with any one or more of FIGS. 1 to 22 may be combined with any of the features, functionality, and alternatives described in connection with any other of FIGS. 1 to 22.

In light of the present disclosure and the above aspects, it is therefore an advantage of the present disclosure to provide a networked beacon apparatus and one or more nodes that provide air/surface purification and/or sterilization based on detected air quality and environmental conditions.

It is another advantage of the present disclosure to provide a networked beacon apparatus that uses ozone to neutralize biological material and UV—C and/or UV-A light to afterwards dissipate the ozone to minimize user irritation from the ozone.

It is yet another advantage of the present disclosure to pause air/surface purification and/or sterilization when an individual is detected within a monitored indoor area.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a diagram of an example purification/sterilization environment, according to an example embodiment of the present disclosure.

FIG. 2 is a diagram of a beacon apparatus, according to an example embodiment of the present disclosure.

FIG. 3 is a diagram of a side view of a detection range of an example proximity detection sensor, according to an example embodiment of the present disclosure.

FIG. 4 is a diagram of a top view of a detection range of the proximity detection sensor, according to an example embodiment of the present disclosure.

FIG. 5 is a diagram of the beacon apparatus of FIG. 2 with a top section in a raised position, according to an example embodiment of the present disclosure.

FIG. 6 is a diagram of the beacon apparatus of FIG. 2 with the top section lowered into a retracted position, according to an example embodiment of the present disclosure.

FIG. 7 is a diagram of a back-side of the beacon apparatus of FIG. 2, according to an example embodiment of the present disclosure.

FIG. 8 is a cut-away diagram showing internal components of the beacon apparatus of FIG. 2, according to an example embodiment of the present disclosure.

FIG. 9 is a diagram illustrating at least some instructions that define conditions under which one or more purification/sterilization modalities are activated, according to an example embodiment of the present disclosure.

FIG. 10 is another diagram illustrating at least some of the instructions that define conditions under which one or more purification/sterilization modalities are activated, according to an example embodiment of the present disclosure.

FIG. 11 is a flow diagram of an example procedure for performing air/surface purification and/or sterilization using the beacon apparatus of FIGS. 1 to 8, according to an example embodiment of the present disclosure.

FIG. 12 is a diagram of a node, according to an example embodiment of the present disclosure.

FIG. 13 is a diagram of a housing of the node of FIG. 12, according to an example embodiment of the present disclosure.

FIG. 14 is a diagram of a program user interface of an application displayed on a user device, according to an example embodiment of the present disclosure.

FIG. 15 is a diagram of an air quality user interface of the application displayed on the user device, according to an example embodiment of the present disclosure.

FIG. 16 is a diagram of a user interface showing beacon apparatuses and nodes in an indoor area, according to an example embodiment of the present disclosure.

FIG. 17 is a diagram of a user interface showing a list of rooms in an indoor area that have at least one beacon apparatus and/or node, according to an example embodiment of the present disclosure.

FIG. 18 is a diagram of a user interface showing a status, air quality, and environmental conditions detected by a beacon apparatus or node, according to an example embodiment of the present disclosure.

FIG. 19 is a diagram of a user interface showing an air quality history detected by a beacon apparatus or node, according to an example embodiment of the present disclosure.

FIG. 20 is a diagram of a user interface of an application that enables a user to schedule one or more modalities to activate on a beacon apparatus or a node, according to an example embodiment of the present disclosure.

FIGS. 21 and 22 are diagrams of the beacon apparatus and the node installed in vehicles/vessels, according to example embodiments of the present disclosure.

### DETAILED DESCRIPTION

An apparatus, method, and system are disclosed herein that provide managed air/surface sterilization and/or purification. The apparatus, method, and system use multiple sterilization/purification modalities to efficiently decontaminate air and surfaces in an indoor environment. The apparatus, method, and system are also configured to reduce or eliminate user irritation by only activing when users are not present. The apparatus, method, and system also reduce or minimize user irritation by causing excess ozone to decay after sterilization/purification is complete.

The example apparatus, method, and system include one or more air quality sensors to determine contamination levels within one or more indoor spaces. Data from the air quality sensors enable the apparatus, method, and system to determine how long one or more air/surface sterilization and/or purification are to be activated. For example, the apparatus, method, and system use data from the air quality sensors to provide UV-C and/or UV-A light, activated oxygen (e.g., ozone), and/or ultrasonic waves for touchless sterilization for the immobilization of containments on surfaces and in the air. The apparatus, method, and system enable a user to specify purification/sterilization modalities based on air quality and/or environmental thresholds. In some embodiments, the apparatus, method, and system may determine and provide one or more purification/sterilization based on detected trends in air quality and when users are present in certain spaces. In some embodiments, data from one or more of the air sensors are used for generating alerts or displaying information about detected concentrations of VOCs and/or gases/vapors including combustible gas/smoke, alcohol vapors, methane, propane, butane, liquefied petroleum, liquid natural gas, carbon monoxide, hydrogen, ozone, ammonia sulfide, or benzene vapor.

As discussed herein, the apparatus, method, and system may include one or more beacon apparatuses in conjunction with one or more nodes. Together, the beacons and nodes form a connected network of sterilization/purification devices that provide managed decontamination of larger indoor spaces, unique spaces, and/or multiple rooms. A beacon apparatus may use air quality data from other beacons and/or nodes to determine which indoor areas should be sterilized and/or purified, which modality should be used, and a duration the modality should be activated. The beacon apparatus may use information about a proximity of users to determine which other beacon apparatuses and/or nodes are to be paused until the users leave the monitored area.

Both the beacon apparatus and nodes are configured to provide UV-C and/or UV-A light, activated oxygen, and/or ultrasonic waves for touchless sterilization/purifications. These modalities can be provided in a compact form factor and do not include filters that have to be cleaned or replaced. Further, the sterilization/purification modalities of the beacon apparatus and the nodes do not use harmful chemicals or require a connection to an online source of water.

Reference is provided herein to purification and sterilization. As discussed herein, purification refers to a process of sanitizing air and/or a surface by neutralizing toxins and anaerobic microbes as an existential threat to human health. Purification performed by the beacon apparatus and nodes is configured to neutralize gases, bacteria, viral or fungal matter, and toxic pathogens. Sterilization refers to a process that removes, kills, or deactivates bacteria, viral or fungal matter, toxic pathogens, and prions. Each of the modalities discussed in reference to the beacon apparatus and nodes may purify and/or sterilize. In some embodiments, an effectiveness of a modality to sterilize and/or purify may be based on environmental factors, such as air temperature and relative humidity. For example, UV-C light may only provide purification at a relatively high humidity but provide sterilization at a relatively low humidity.

Purification/Sterilization Environment

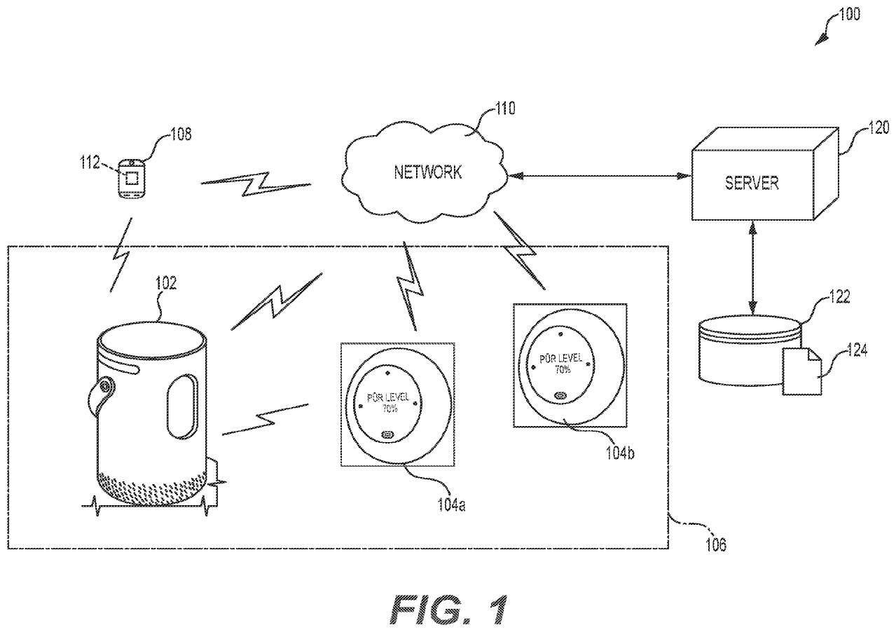

FIG. 1 is a diagram of an example purification/sterilization environment 100, according to an example embodiment of the present disclosure. The environment 100 includes at least one beacon apparatus 102. The environment 100 also includes two nodes 104a and 104b. In other embodiments, the environment 100 may omit the nodes 104a, only include one node 104, or include a plurality of nodes 104. Further, the environment 100 may include additional beacon apparatus 102.

The beacon apparatus 102 and nodes 104 are located within an indoor area 106. As described herein, the indoor area 106 may include one or more rooms of a residence, an office, a school, a vehicle, or other space that needs purification and/or sterilization. For example, the indoor area 106 may include a conference center, a hotel, a stadium, a museum, a gym, a cruise ship, an airplane, a bus, a train, etc.

In the illustrated example, the beacon apparatus 102 is communicatively coupled to a user device 108. The beacon apparatus 102 may be connected to the user device 108 via a network 110, which may include any local area network (“LAN”), wireless LAN, Wi-Fi, wide area network (“WAN”) such as the Internet, a cellular network, or combinations thereof. The beacon apparatus 102 may be locally connected to the user device 108 via a local connection, such as via a universal serial bus (“USB”) connection or a Molex® connection, or a wireless interface, such as a Bluetooth®, Zigbee®, or a Near-Field Communication (“NFC”) connection.

The nodes 104 are communicatively coupled to the user device 108 and/or the beacon apparatus 102. The connection maybe via the network 110 and/or a short range wireless connection using Bluetooth® or Zigbee®, for example. If communication is via Wi-Fi, Bluetooth®, or Zigbee®, the nodes 104 and the beacon apparatus 102 are configured to form a local network, which may include a mesh or ad hoc network to enable communication therebetween.

The example user device 108 is configured to receive status and/or air quality data from the beacon apparatus 102 and/or the nodes 104. The user device 108 is also configured to transmit instructions to the beacon apparatus 102 and/or the nodes for programming, initiating, or stopping purification/sterilization. The user device 108 includes a processor, a memory, and an interactive display screen. The user device 108 may include any smartphone, tablet computer, laptop computer, desktop computer, workstation, server, etc. The memory of the user device 108 is configured to store instructions that define an application 112. Execution of the instructions by the processor of the user device 108 causes the application 112 to be operated according to the description provided herein.

The application 112 is configured to manage status and air quality information for display within one or more user interfaces. The application 112 may compile air quality and/or status trends to show contamination history of a monitored indoor area to a user. The application 112 also includes one or more user interfaces for activating one or more sterilization/purification modalities of the beacon apparatus 102 and/or the nodes 104. The application 112 may, for example, enable a user to set an activation schedule and/or one or more conditions as to when sterilization/purification is to occur. In addition, the application 112 may provide a list of graphical map showing locations of the nodes 104 and the beacon apparatus 104. The list of graphical map locations may also display an indicator of air quality and/or a status. Selection of a device causes the application 112 to display another user interface with additional status or air quality data for the selected node 104 or beacon apparatus 102. The application 112 may also display alert notification after detecting that air quality data exceeds a threshold.

The environment 100 of FIG. 1 also includes a server 120 communicatively coupled to a memory device 122. The server 120 is coupled to the user device 108, the beacon apparatus 102, and/or the nodes 104 via the network 110. The server 120 may include any workstation, cloud computing environment, and/or distributed computing environment.

The server 120 is configured to receive status and/or air quality information, which may be used for analytics. For example, the server 120 may use status and/or air quality information associated with a user to aggregate air quality trends for display in the application 112. The server 120 may compare a user's air quality trends to other users to determine recommendations for activating one or more purification/sterilization modalities of the beacon apparatus 102 and/or the nodes 104. Further, in instances where the user device 108 is out of Wi-Fi and Bluetooth® range of the indoor area 106, the server 120 is configured as a bridge between the user device 108 and the beacon apparatus 102 and/or nodes 104. For example, the server 120 receives status and air quality data from the beacon apparatus 102 and/or nodes 104 via one or more application programmable interfaces (“APIs”) and transmits the status and air quality data to the application 112 via one or more other APIs for population in one or more template user interfaces. Further, the application 112 may transmit programming or operational instructions to the server 120, which relays the instructions to the beacon apparatus 102 and/or the nodes 104.

The example server 120 is configured to register the application 112 to the beacon apparatus 102 and/or the nodes 104 via a registration process. Registration association information is stored in a data structure 124 in the memory device 122, and may include an application identifier, user registration information, and/or network identifiers/addresses for the user device 108, the beacon apparatus 102, the nodes 104, and/or networking/gateway equipment at the indoor area 106 that provide Internet connectivity to for the beacon apparatus 102 and the nodes 104. The memory device 122 may include any memory including a solid state drive, a hard disk drive, flash memory, etc.

Beacon Apparatus Embodiment

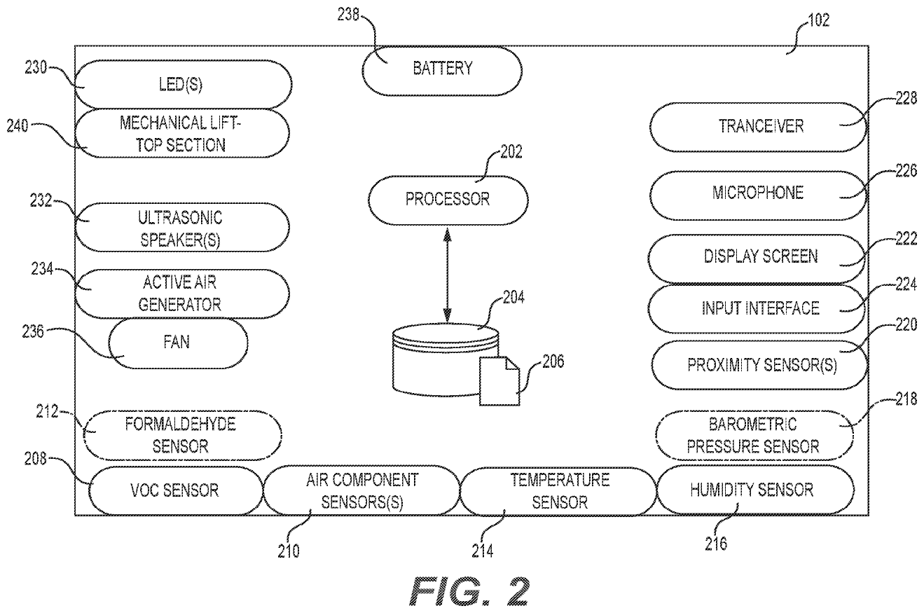

FIG. 2 is a diagram of the beacon apparatus 102 of FIG. 1, according to an example embodiment of the present disclosure. The example beacon apparatus 102 includes a processor 202 and a memory device 204 storing instructions 206. Execution of the instructions 206 by the processor 202 causes the beacon apparatus 102 to perform the operations described herein. The instructions 206 may also specify one or more conditions for activating one or more purification/sterilization modalities, as described herein. In some embodiments, at least operations may be performed by another component rather than the processor 202. For example, a VOC sensor 208 may include a microcontroller and/or application specific integrated circuit (“ASIC”) configured to detect a gas concentration and output digital data indicative of a gas type and/or concentration.

The example beacon apparatus 102 includes one or more sensors for detecting containments and/or air quality. The sensors include one or more VOC sensor(s) 208, one or more air component sensors 210, and/or a formaldehyde sensor 212. The VOC sensor 108 may include a Sensirion® SGPC3 sensor for detecting a presence and/or concentration of VOCs within ambient air. The formaldehyde sensor 212 is configured to measure aerosol formaldehyde in a range between 1 to 100 parts per million (“ppm”). The air component sensors 210 are configured to provide for detection of concentrations of certain gases including one or more of ozone (i.e., O³), carbon dioxide, combustible gas/smoke, alcohol vapors, methane, propane, butane, liquefied petroleum, liquid natural gas, carbon monoxide, hydrogen, ozone, ammonia sulfide, or benzene vapor. In some embodiments, the air component sensor 210 may include a biosensor for detecting a presence and/or concentration of microbes, such as bacteria. The sensors 208 to 212 periodically transmit digital data to the processor 202 that is indicative of a presence and/or concentration of a certain gas. Alternatively, the sensors 208 to 212 may transmit an analog signal that is indicative of a gas concentration.

An ozone air component sensor 210 is configured to measure ozone levels before, during and/or after purification/sterilization modalities have been activated. A carbon dioxide air component sensor 210 is configured to provide data indicative of a space occupancy as a proxy for a density of individuals in a room. In some embodiments, the application 112, the processor 202, and/or the server 120 is configured to use carbon dioxide data as an input for setting purification/sterilization levels based on estimated occupancy density. For instance, additional or longer purification may be provided in response to detecting greater concentrations of individuals in an area, as indicated by greater carbon dioxide levels.

The example beacon apparatus 102 also includes sensors for detecting environmental conditions. The sensors include a temperature sensor 214, a relative humidity sensor 216, and/or a barometric pressure sensor 218. The temperature sensor 214 is configured to measure an ambient air temperature between a range of −40° C. to 125° C., for example. The relative humidity sensor 216 is configured to measure a relative humidity between 0 to 100%. The barometric pressure sensor 218, which is optional, is configured to measure an atmospheric pressure within the indoor area 106. The sensors 214 to 218 are configured to transmit either digital or analog data indicative of a temperature, relative humidity, and/or barometric pressure.

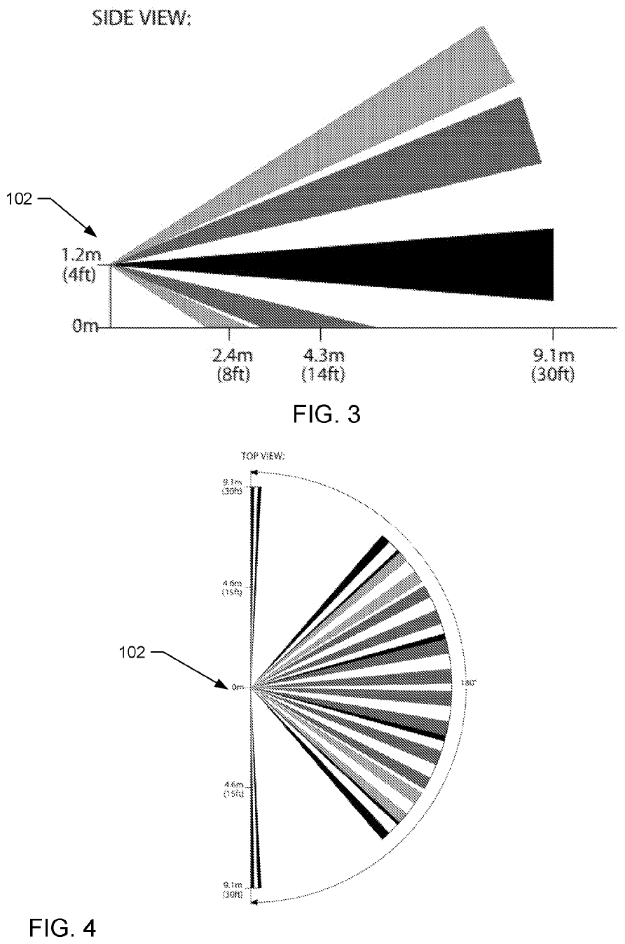

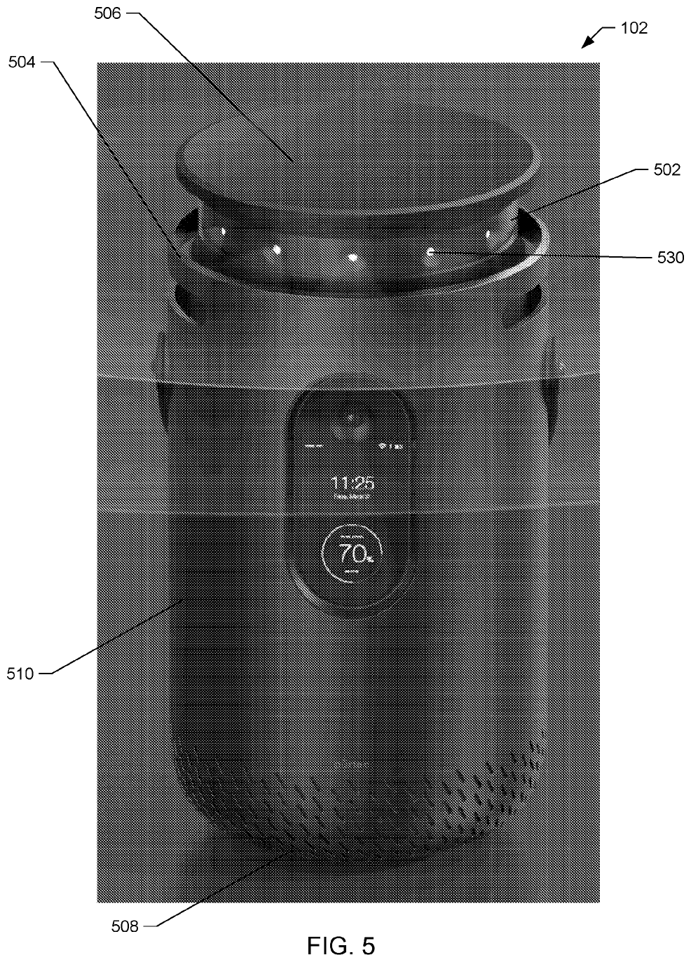

The illustrated beacon apparatus 102 of FIG. 2 includes one or more proximity detection sensors 220 to detect a presence of individuals. The proximity sensors 220 may include, for example, passive infrared (“PIR”) sensors that measure infrared light radiating from objects within a field of view. The proximity sensors 220 have a detection range between 10 and 30 feet and an ultra-wide field of view. FIG. 3 is a diagram of a side view of a detection range of an example PIR proximity sensor 220, according to an example embodiment of the present disclosure. FIG. 4 is a diagram of a top view of a detection range of an example PIR proximity sensor 220, according to an example embodiment of the present disclosure. As shown, when the beacon apparatus 102 is place at a height of 4 feet, a PIR proximity sensor 220 has a range up to 30 feet and a field-of-view of approximately 180°. The beacon apparatus 102 includes at least two, and preferably four or five, proximity sensors 220 to provide overlapping proximity detection. The proximity sensor 220 is configured to transmit a digital message and/or an analog signal to the processor 202 after detecting a presence of an individual or object. The proximity sensor 220 may be calibrated or self-calibrate for a given indoor area 106 to account for furniture and other inanimate objects.

The example beacon apparatus 102 of FIG. 2 is configured to receive inputs from a user. The apparatus 102 includes a display screen 222 and an input interface 224. The display screen 222 may include a liquid crystal display and is configured to display a graphical user interface that provides information indicative of monitored air quality and/or an operational status. The processor 202 may cause at least a portion of the display screen 222 to change color based on a detected air quality. For example, red/yellow colors may be displayed in a background to indicate many air containments while a green/blue background is shown when there are few detected air containments. The display screen 222 may also display interfaces to enable a user to enter a setting or activate a modality of the beacon apparatus 102. The input interface 224 may include a touchscreen and/or one or more buttons. The input interface 2224 is configured to receive a user input to, for example, select and/or schedule a purification/sterilization modality. The input interface 224 may also include a power switch.

In some embodiments, the beacon apparatus 102 may include a microphone 226 for receiving voice commands/inputs from a user. The processor 202 or a voice controller provided with the microphone 226 that converts voice commands into digital messages. The processor 202 is configured to analyze the digital messages to determine an input command. The memory device 204 may store a library of supported voice input commands that causes the processor 202 to perform a certain operation. For example, the processor 202 may actuate a certain purifications/sterilization modality after receiving a command identifying the modality (i.e., “Begin ozone and cleaning light” or “Start Purification”). A user may also use the microphone 226 to verbally schedule times and/or conditions upon which one or more purifications/sterilization modalities are to be activated.

The example beacon apparatus 102 further includes one or more transceivers 228. The example transceiver 228 may include one or more antennas to provide wireless communication via Wi-Fi, Bluetooth®, Zigbee®, etc. The transceiver 228 may also support one or more wired data connections, such as a data connection via the USB protocol. In some embodiments, the transceiver 228 is configured to support Internet of Things (“IoT”) connectivity with the server 120, other registered beacon apparatuses 102, and/or registered nodes 104.

The example beacon apparatus 102 also includes components that provide the purification/sterilization modalities discussed herein. The components include one or more UV-C and/or UV-A LED(s) 230, one or more ultrasonic speaker(s) 232, and an active air (oxygen) generator 234. In some embodiments, the LEDs may be provided around a perimeter of the beacon apparatus 102 to provide 360° purification/sterilization. At least some of the LEDs are configured to emit light in the 250 to 270 nanometer (“nm”) wavelength range, preferably between 254 to 265 nm to inactivate viral material. In some embodiments, other of the LEDs are configured to emit light in the 390 to 420 nm wavelength range, preferably in the 400 to 410 nm range to inactivate bacteria. The LEDs may have an output power of four watts and a viewing angle between 90° and 150°, preferably around 130°.

The one or more ultrasonic speakers 232 are configured to emit acoustic waves to aggregate suspended biological and/or chemical material and inactivate such. The speakers 232 may include a tweeter or a piezo loudspeaker with a maximum power of 300 watts and emit a waveform with a frequency between 20 and 80 kHz, preferably around 40 kHz. The speakers 232 are configured to provide acoustic waves with a sound pressure level between 80 and 150 dB, preferably around 105 dB or 120 dB and an angle of radiation between 45° and 180°, preferably between 150° and 160°. The beacon apparatus 102 may include more than one speaker 232 to provide 360° of coverage.

The active air generator 234 is configured to generate ozone at a rate between 4 to 20 grams/hour, preferably around 10 grams/hour. The active air generator 234 may include an ozone ionizer plate that operates at a frequency between 18 to 20 kHz. The active air generator 234 catalyzes the creation of ozone from ambient air. The beacon apparatus 102 includes a fan 236 to circulate the created ozone. In some embodiments, the processor 202 may activate the fan 236 periodically to cause ambient air to flow over the sensors 208 to 218 to perform an air quality or environment measurement.

Together, the LED(s) 230, the one or more ultrasonic speaker(s) 232, and the active air generator 234 are configured to provide air/surface purification and/or sterilization for an indoor area 106 that is between 150 to 1500 ft². The LED(s) 230, the one or more ultrasonic speaker(s) 232, and the active air generator 234 may provide 99% microbe immobilization within 20 seconds for a six foot radius around the beacon apparatus 102. The LED(s) 230, the one or more ultrasonic speaker(s) 232, and the active air generator 234 may provide 99% microbe immobilization within 45 minutes for a six foot radius and 99% microbe immobilization within 60 minutes for a twelve foot radius around the beacon apparatus 102.

The example beacon apparatus 102 may include a battery 238 to provide power for the processor 202 and the other components 204 to 236 discussed above. The battery 238 is configured to be rechargeable via a wired or wireless connection. Further, the battery 238 may include an alternating current converter to enable power to be received directly from an electrical outlet.

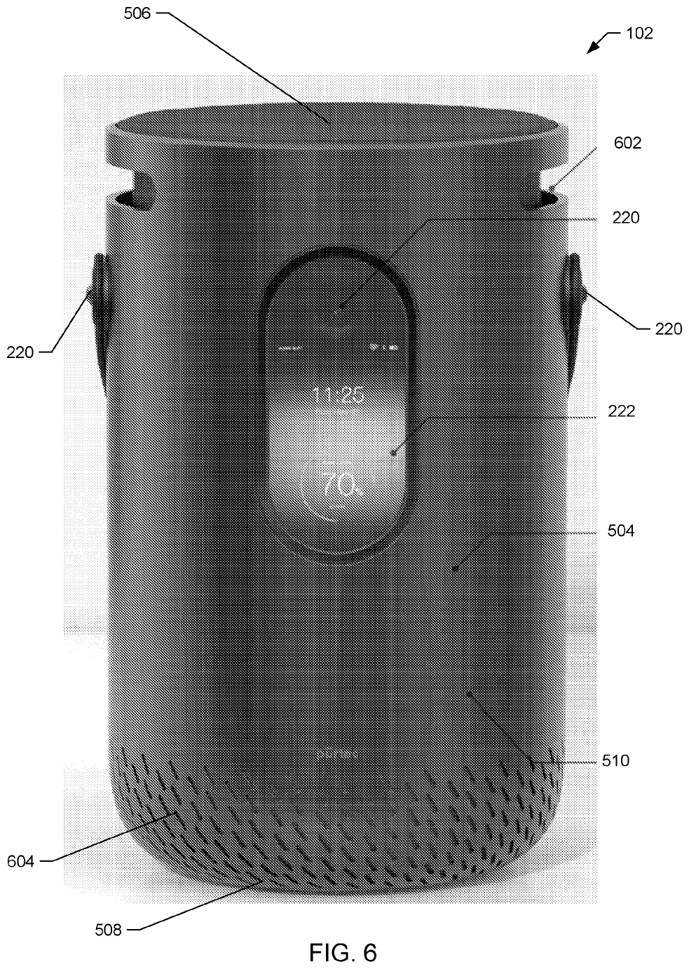

In some embodiments, the beacon apparatus 102 of FIG. 2 may include a mechanical lift top section 240 to enable the LEDs to be moved between a raised position and a retracted position. FIG. 5 is a diagram of the beacon apparatus 102 with a top section 502 in a raised position, according to an example embodiment of the present disclosure. In the illustrated example, the beacon apparatus 102 includes a housing 504 having a top side 506, a bottom side 508, and a cylindrical face 510 that is located between the top side 506 and the bottom side 508. The top side 506 of the housing 504 includes the top section 502, which is configured to move up and down with respect to the housing 504. The top section 502 has a cylindrical shape. A plurality of the LEDs 230 is placed around a circumference of the top section 502. In the illustrated example, at least ten LEDs 230 are placed around the top section 502.

The mechanical lift top section 240 includes a motor configured to provide mechanical actuation to raise and lower the top section 502, including the LEDs 230. The motor is connected to the top section 502 via an actuator arm. The motor is configured to cause the actuator arm to raise and lower the top section 502 with respect to the housing 504 such that the plurality of the LEDs 230 are exposed when the top section 502 is in the raised position and hidden from view when the top section 502 is in the retracted position. The processor 202 is configured to cause the motor to raise the top section 240 when the LEDs 230 are to be activated and cause the motor to lower the lower the top section 240 when the LEDs 230 are turned off.

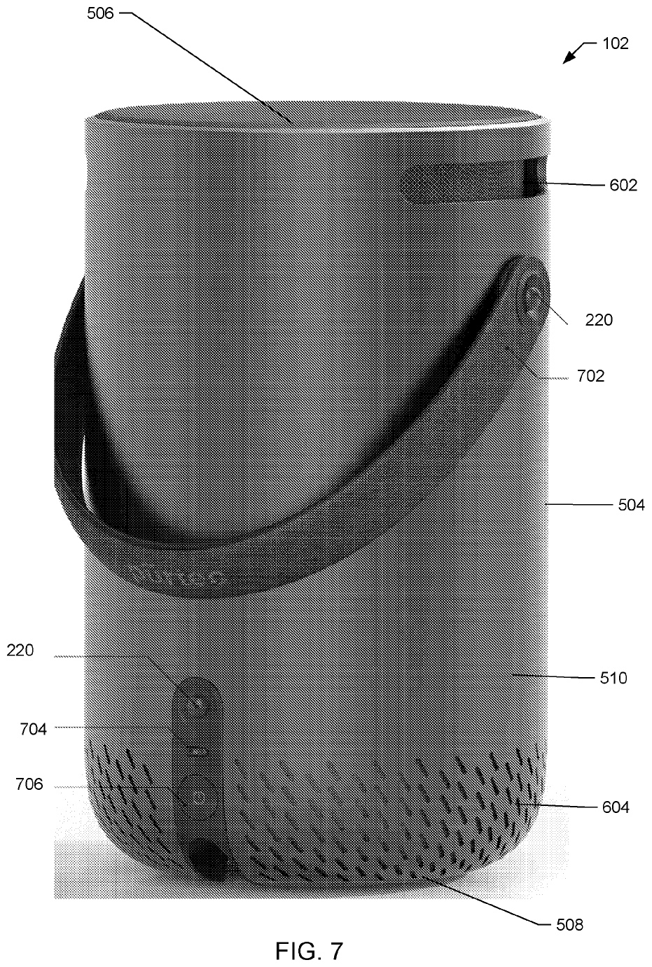

FIG. 6 is a diagram of the beacon apparatus 102 with the top section 502 lowered into the retracted position, according to an example embodiment of the present disclosure. In the retracted position, the top section 502 is located within the housing 504, which prevents the LEDs 230 from being visible. In the retracted position, the beacon apparatus 102 has a more streamlined appearance while hiding the mode distracting LEDs 230. In some embodiments, the mechanical lift top section 240 is not present and the LED(s) 230 are instead provided on the housing 504.

FIG. 6 also shows that the cylindrical face 510 of the housing 504 includes a first vent 602 located adjacent to the top side 506 and a second vent 604 located adjacent to the bottom side 508. The vents 602 and 604 are formed in windows or holes of the housing 504. When the fan 236 is active, ambient air is pulled in through the second vent 602 and expelled through the first vent 602. This air flow enables the ambient air to flow over the sensors 208 to 218 to determine air quality and/or environmental conditions. Also, when the active air generator 234 is active, the flow of air is used to supply oxygen needed for catalyzing ozone ionizers. The ozone is then expelled through the vent 602.

The housing 504 is shown in a cylindrical shape and is comprised of metal, such as anodized aluminum. In other embodiments, the housing 504 may have a cube, rectangular prism, or pyramidal shape. Further, the housing 504 may include other materials, such as plastic, composites, wood, or combinations thereof.

FIG. 6 also shows locations of three proximity sensors 220. A first proximity sensor is provided with the display screen 222. Two other proximity sensors 220 are provided where a handle connects to the housing 504. The proximity sensors 220 are located approximately 90° apart to provide 360° proximity detection of individuals.

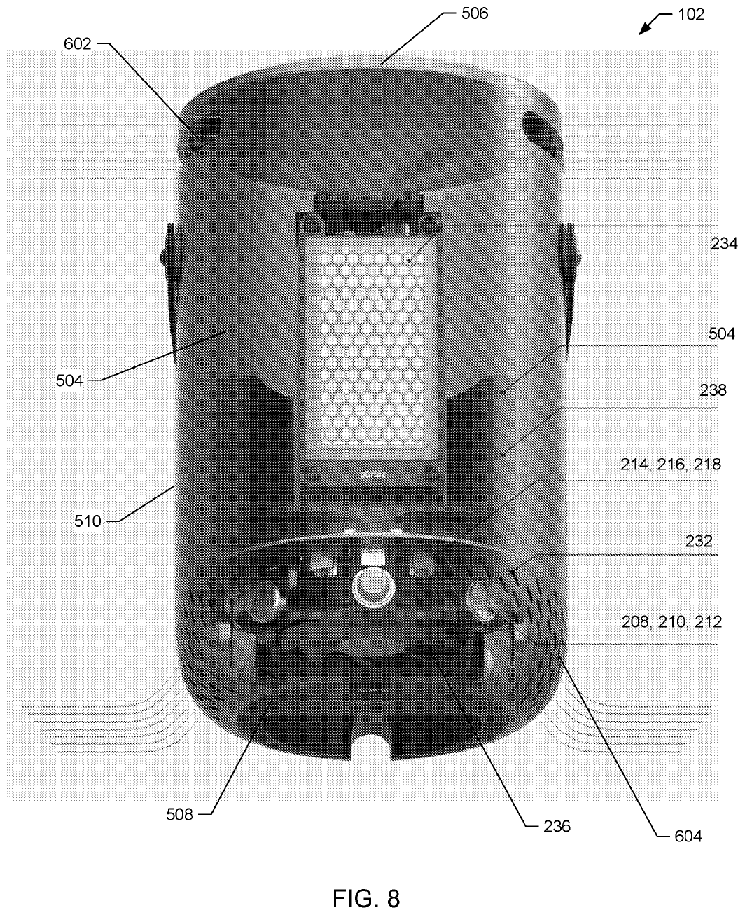

FIG. 7 is a diagram of a back-side of the beacon apparatus 102, according to an example embodiment of the present disclosure. The illustrated example shows the proximity sensor 220 located at a connection point of a handle 702 to the cylindrical face 510 of the housing 504. Another proximity sensor 220 is located along a bottom of the cylindrical face 510, adjacent to Wi-Fi enable button 704 and a power button 706. In other embodiments, the proximity sensors 220 are located in an array around an upper-circumference of the housing 504.

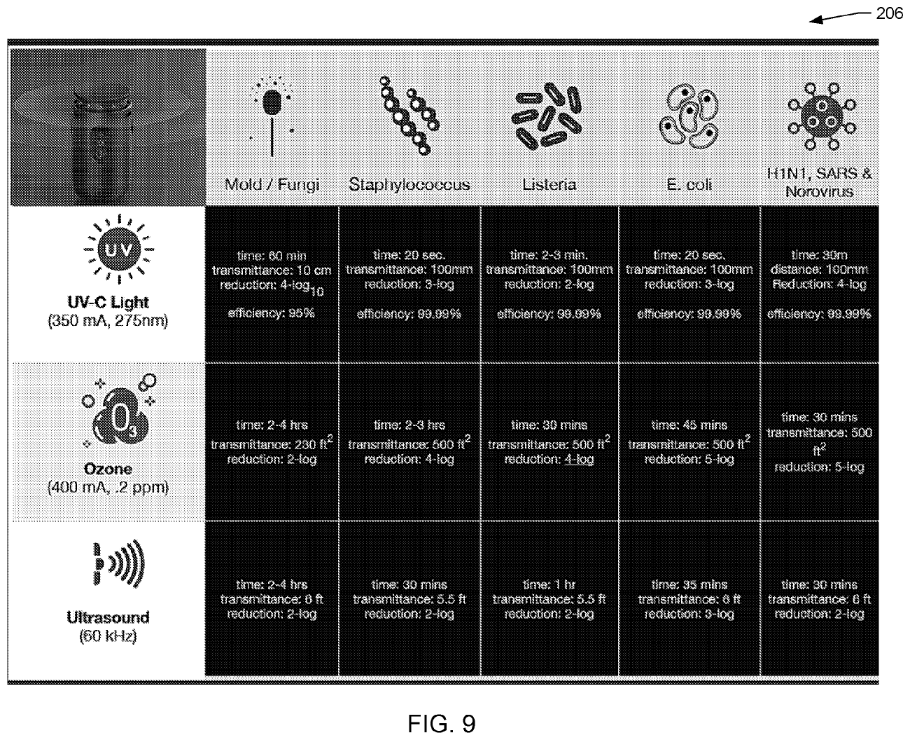

FIG. 8 is a cut-away diagram showing internal components of the beacon apparatus 102, according to an example embodiment of the present disclosure. The diagram shows air flow when the active air generator 234 is turned on. As shown, ambient air enters the second vent 604 and is pulled upward through the housing 504 via the fan 236. The air passes over ozone ionizers of the active air generator 234, which causes ozone to form. The air with the newly formed ozone is then expelled through the first vent 602 due to air flow formed by the fan 236. The air flow also passes over the air quality sensors 208 to 212, which are located inside the housing 504 along the air flow path. The air flow also passes over the environment sensor 214 to 218, which are also located along the air flow. It should be appreciated that the active air generator 234 is located downstream from the sensors 208 to 218, which prevents newly created ozone from inadvertently affecting air quality measurements.

FIG. 8 also shows that the ultrasonic speakers are located adjacent to the bottom side 508 within the housing 504. The battery 238 is located between the sensors 208 to 218 and the active air generator 234 to provide a further barrier for the newly created ozone, without affecting air flow. The flow of air over the battery 238 also provides cooling, thereby extending battery life.

Purification/Sterilization Algorithm Embodiments

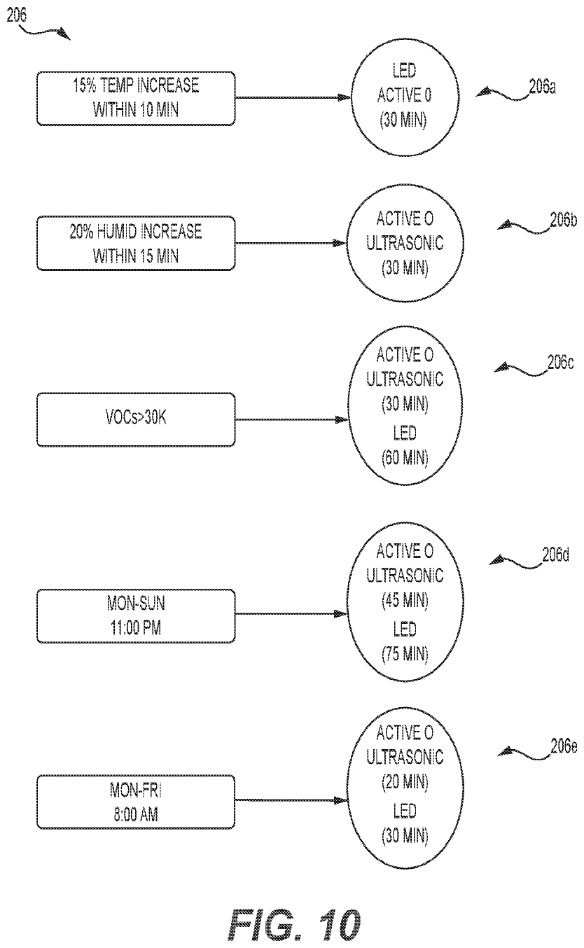

As discussed above in connection with FIG. 2, the memory device 204 of the beacon apparatus 102 includes instructions 206 that define operations performed by the processor 202. The example instructions 206 may also define conditions under which the processor 202 is to activate one or more purification/sterilization modalities discussed herein. FIG. 9 is a diagram illustrating at least some of the instructions 206 that define conditions under which one or more purification/sterilization modalities are activated, according to an example embodiment of the present disclosure. A top row of a table representing the instructions 206 identifies different types of common biological material including mold/fungi, staphylococcus, listeria, E. coli, and the H1N1 virus. In other examples, the instructions 206 may identify other biological material such as herpes, rhinovirus, and influenza. A first column of the instructions 206 identifies the purification/sterilization modalities including use of the LED(s) 230, active air generator 234, and the ultrasonic speaker(s) 232.

In instances where the air component sensors 210 provide for the detection of the listed biological material, the processor 202 is configured to activate the purification/sterilization modalities based on the detected biological material. For instance, the air component sensor 210 may include one or more hyper-spectral imaging devices for detection of microbes and/or ribonucleic acid (“RNA”) material for viral detection in air or on surfaces. In these instances, the instructions 206 may specify a certain concentration or count threshold before the modalities are activated. Alternatively, the instructions 206 may specify that the modalities are activated when any type of the biological material is detected by the air component sensor 210. In an example, the processor 202 receives a signal or message from the air component sensor 210 indicative of a detection of mold/fungi. In response, the processor 202 uses the instructions 206 to determine that the LED(s) 230 are to be activated for 60 minutes, which provides a 4-log₁₀ reduction in mold/fungi with a 95% efficiency. In addition, the processor 202 uses the instructions 206 to determine that the active air generator 234 is to be active for two to four hours, which provides a 4-log₁₀ reduction for 230 ft². Moreover, the processor 202 uses the instructions 206 to determine that the ultrasonic speakers 232 are to be active for two to four hours, which provides a 2-log₁₀ reduction. In this example, the processor 202 causes the LEDs 230 to activate for 60 minutes while causing the active air generator 234 and the ultrasonic speakers 232 to be active for two to four hours. The processor 202 may receive periodic signals from the air component sensor 210 to confirm mold/fungi are no longer detected or detected at a concentration/count below a threshold.

In another example, the different types of biological material may be selected as purification/sterilization options on a user interface of the application 112. Selection of a biological material type causes the processor 202 to perform the corresponding purification/sterilization specified by the corresponding instructions 206 in FIG. 9. In this example, the beacon apparatus 102 may not include air component sensors 210 that provide for the detection of biological material, but enables a user to select which types of purification/sterilization is to be performed to neutralize a potential presence of the biological material. For example, after having a gathering and later finding out that a guest had the H1N1 virus, the user selects the H1N1 option via the user interface, which causes the processor 202 to activate the LEDs, the active air generator 234 and the ultrasonic speakers 232 for 30 minutes to neutralize any potential H1N1 virus left by the guest.

In another example, the beacon apparatus 102 may not detect biological material, but instead use signals from the temperature sensor 214, humidity sensor 216, and/or VOC sensor 208 to determine conditions that are favorable to certain biological material. In response, the processor 202 is configured to activate the sterilization/purification modalities corresponding to the predicted biological material.

As discussed above, at least some of the LEDs 230 may emit light around a wavelength of 254 nm while other LEDs 230 emit light around a wavelength of 405 nm. The 254 nm LEDs 230 provide about 75 to 130 milliJoules (“mJ”)/cm² of energy to neutralize viruses and destroy ozone. The 405 nm LEDs 230 provide about 1.8 to 5 Joules (“J”)/cm² of energy to neutralize bacteria. The UV light disrupts cell RNA of the biological material. In some instances, the processor 202 may activate only the 254 nm LEDs 230 or the 405 nm LEDs 230 based on whether bacteria or viral material is to be neutralized. The active air generator 234 outputs 49 to 96 milligrams (“mg”)/m³ of ozone, which oxidizes cell membranes to neutralize biological material. Further, the ultrasonic waves of the speakers 232 disrupt bacterial capsules to provide neutralization.

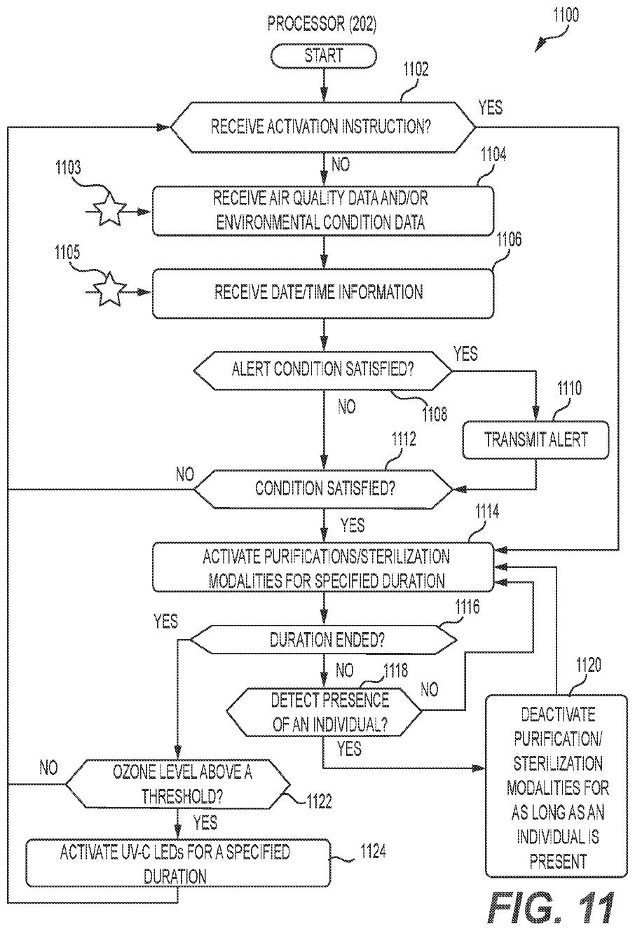

FIG. 10 is another diagram illustrating at least some of the instructions 206 that define conditions under which one or more purification/sterilization modalities are activated, according to an example embodiment of the present disclosure. The instructions 206 shown in FIG. 10 may be created based on user input via the input interface 224 of the beacon apparatus 102 and/or the application 112 of the user device 108. To create the instructions 206, a user selects a condition and a corresponding one or more purification/sterilization modalities.

As shown in FIG. 10, a condition may include one or more air quality metrics. For example, the instruction 206a specifies a condition corresponding to a 15% increase in temperature within ten minutes. Additionally, instruction 206b specifies a condition corresponding to a 20% increase in humidity within 16 minutes, and instruction 206c specifies a condition corresponding VOCs exceeding 30 k. The instruction 206a specifies that if the condition is satisfied, the processor 202 is to activate the LEDs 230 and the active air generator 230 for 30 minutes. The instruction 206b specifies that if the condition is satisfied, the processor 202 is to activate the active air generator 230 and the ultrasonic speakers 232 for 30 minutes. Further, instruction 206c specifies that if the condition is satisfied, the processor 202 is to activate the LEDs 230 for 60 minutes and the active air generator 230 and the ultrasonic speakers 232 for 30 minutes.

Also as shown in FIG. 10, a condition may be based on a date/time duration. For example, instruction 206d specifies that the processor 202 is to activate the LEDs 230 for 75 minutes and the active air generator 230 and the ultrasonic speakers 232 for 45 minutes every day of the week starting at 11:00 PM. Instruction 206e specifies that the processor 202 is to activate the LEDs 230 for 30 minutes and the active air generator 230 and the ultrasonic speakers 232 for 20 minutes every week day starting at 8:00 AM. The example processor 202 compares a current date/time, air quality data, and/or environmental air data to determine which of the conditions specified by the instructions 206 are satisfied. The processor 202 then performs the specified purification/sterilization modalities of the satisfied conditions.

It should be appreciated that FIG. 10 shows only a small subset of possible conditions. Other conditions may be based on the detection of certain gases above a concentration including ozone, carbon dioxide, combustible gas/smoke, alcohol vapors, methane, propane, butane, liquefied petroleum, liquid natural gas, carbon monoxide, hydrogen, ozone, ammonia sulfide, or benzene vapor. Other conditions may be satisfied based on individual presence detection. For example, the instructions 206 may specify that the processor 202 is to activate one or more purification/sterilization modalities after a user has left a monitored area if the user was in the area for at least 30 minutes. Yet other conditions may be based on a combination of environmental conditions such as humidity and temperature values in addition to air quality measurements made by the VOC sensor 208 and/or the air component sensors 210.

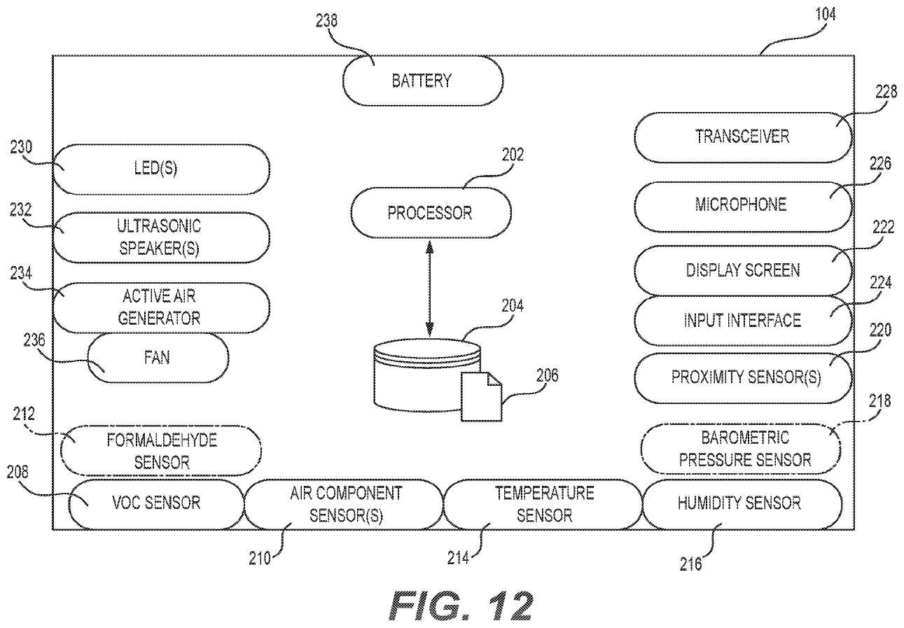

FIG. 11 is a flow diagram of an example procedure 1100 for performing air/surface purification and/or sterilization using the beacon apparatus 102 of FIGS. 1 to 8, according to an example embodiment of the present disclosure. Although the procedure 1100 is described with reference to the flow diagram illustrated in FIG. 11, it should be appreciated that many other methods of performing the steps associated with the procedure 1100 may be used. For example, the order of many of the blocks may be changed, certain blocks may be combined with other blocks, and many of the blocks described may be optional. In an embodiment, the number of blocks may be changed based on conditions used for activating certain purification/sterilization modalities. The actions described in the procedure 1100 are specified by one or more instruction and may be performed among multiple devices including, for example, the beacon apparatus 102, the node 104, the application 112, and/or the server 120.

The example procedure 1100 begins when the processor 202 of the beacon apparatus 102 determines if an activation instruction has been received via the input interface 224 and/or the application 112 (block 11020). The activation instruction is indicative that the beacon apparatus 102 is to immediately begin one or more purification and/or sterilization modalities. If an activation instruction is received, the processor 202 activates the specified purification/sterilization modality for a specified duration or until a user provides a deactivation instruction (block 2114).

If an activation instruction is not received, the processor 202 receives air quality data and/or environmental condition data 1103 from one or more of the sensors 208 to 218, as discussed above (block 1104). The processor 202 also receives date/time data 1105 from an internal clock (block 1106). The processor 202 next compares the data 1103 and/or 1105 to one or more alert conditions stored in the instructions 206 of the memory device 204 (block 1108). The alert conditions may specify one or more air quality thresholds. In some embodiments, the processor 202 adds the newly received data 1103 to 1105 to a trend history of the data 1103 to 1105 and compares an average of a recent trend to one or more thresholds. Exceeding a threshold indicates the presence or excess concentration of an undesirable gas that may require attention from a user. The gases may include ozone, carbon dioxide, combustible gas/smoke, alcohol vapors, methane, propane, butane, liquefied petroleum, liquid natural gas, carbon monoxide, hydrogen, ozone, ammonia sulfide, or benzene vapor.

If an alert condition is satisfied, the processor 202 causes an alert to be transmitted (block 1110). The alert may be displayed on the display screen 222 of the beacon apparatus 102. The alert may also be displayed as an alert notification by the application 112 on the user device 108. The alert may identify the condition that triggered the alert and/or provide a visual indication regarding a severity. In some embodiments, the determination as to whether an alert is to be generated is performed by the server 120 and/or the application 112 after receiving data 1103 and/or 1105 from the processor 202.

After determining whether an alert is to be transmitted, the example procedure 1100 continues by determining whether one or more air quality and/or day/time conditions are satisfied (block 1112). If a condition is not satisfied, the processor 202 returns to block 1102 to check if an activation instruction was received from a user. However, if at least one condition is satisfied, the processor 202 determines which purification and/or sterilization modalities are to be activated and a duration each is to be active (block 1114). For example, the processor 202 may activate the LEDs 230, the ultrasonic speakers 232, and/or the active air generator 234 based on the instruction 206 corresponding to the satisfied condition.

The example processor 202 then determines if a duration of a purification/sterilization modality has ended (block 1116). If a duration has not ended, the processor 202 determines if a signal or message is received from the proximity sensor(s) 220 (block 1118). If an individual is detected, the processor 202 is configured to deactivate the purification/sterilization modalities (block 1120). The processor 202 keeps the modalities deactivated for as long as the individual is detected by the one or more proximity sensors 220. After this time, the processor 202 reactivates the purification and/or sterilization modalities for the remaining duration (block 1114). In some instances, the instructions 206 cause the processor to wait a certain time duration after when the individual is no longer detected before activation of the modalities can begin. The time duration may be any time between one second and a few hours. In some instances, the time duration may be defined by a user and stored as a condition for an instruction 206.

If an individual is not detected, the processor 202 causes the modalities to continue operating until an end of the specified duration. The processor 202 then determines, using data from the VOC sensor 208 and/or the air component sensor 210 whether an ozone level in ambient air is above a threshold (block 1122). If the ozone level is above the threshold, the processor 202 activates the LEDs 230 until a concentration or amount of the ozone falls below the threshold (block 1124). In some embodiments, the processor 202 does not receive data indicative of ozone and instead activates the LEDs 230 for a duration based on how long the active air generator 234 was active. For example, the processor 202 may activate the LEDs 230 for 15 minutes to decompose ozone for every 30 minutes that the active air generator 234 was active. The example procedure 1100 returns to block 1102 to determine if an activation instruction is received 1102 and/or check for modality/alert conditions.

Node Embodiment

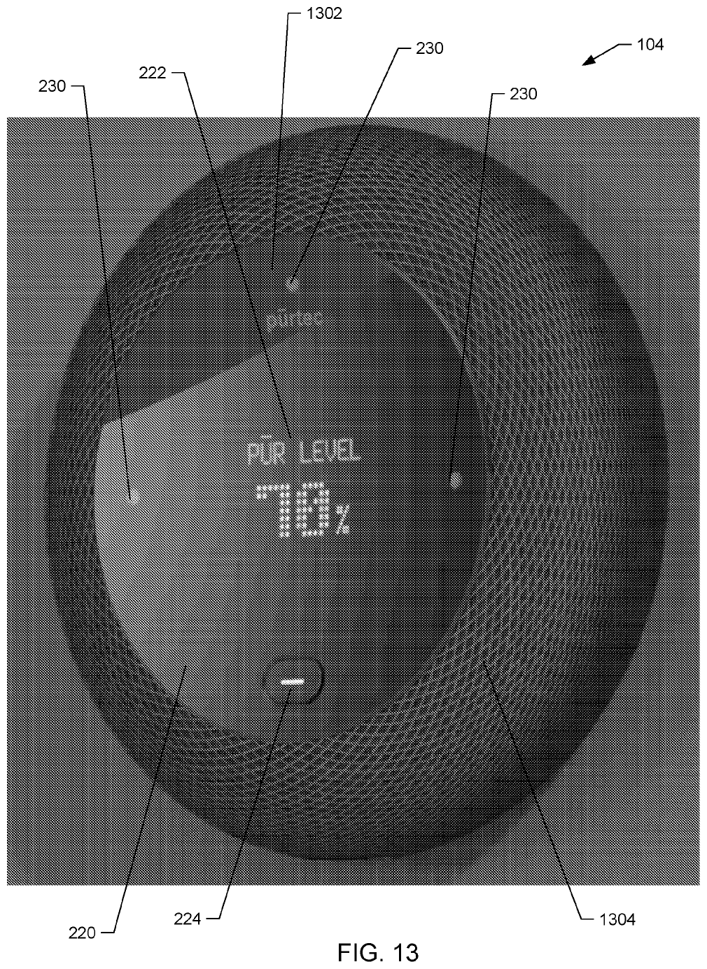

FIG. 12 is a diagram of the node 104 of FIG. 1, according to an example embodiment of the present disclosure. The node 104 is configured to have many of the same features as the beacon apparatus 102, however scaled down to fit into a disk-like form factor that can be hung on a wall or a ceiling. Similar to the beacon apparatus 102, the node 104 includes a processor 202, a memory device 204 storing instructions 206, at least one VOC sensor 208, one or more air component sensors 210, a formaldehyde sensor (optional) 212, a temperature sensor 214, a humidity sensor 216, a barometric pressure sensor (optional) 218, one or more proximity sensors 220, a display screen 222, an input interface 224, a microphone 226, a transceiver 228, one or more LEDs 230, ultrasonic speakers 232, and active air generator 234, a fan 236, and a battery 238. Unlike the beacon apparatus 202, the node 204 does not include a mechanical lift 240.

The components 202 to 238 are configured to perform the same operations as described above in connection with the beacon apparatus 102. The active air generator 234 and fan 236 configured to be smaller and output ozone, for example, in 0.5 mg/liter bursts during 15 minute increments. Further, the display screen 222 may include a pixel-based display rather than a liquid crystal display. It should be appreciated that the node 104 may be operational without needing a connection to a beacon apparatus 102. Instead, the node 104 may be a standalone device for smaller spaces between 10 ft² and 150 ft².

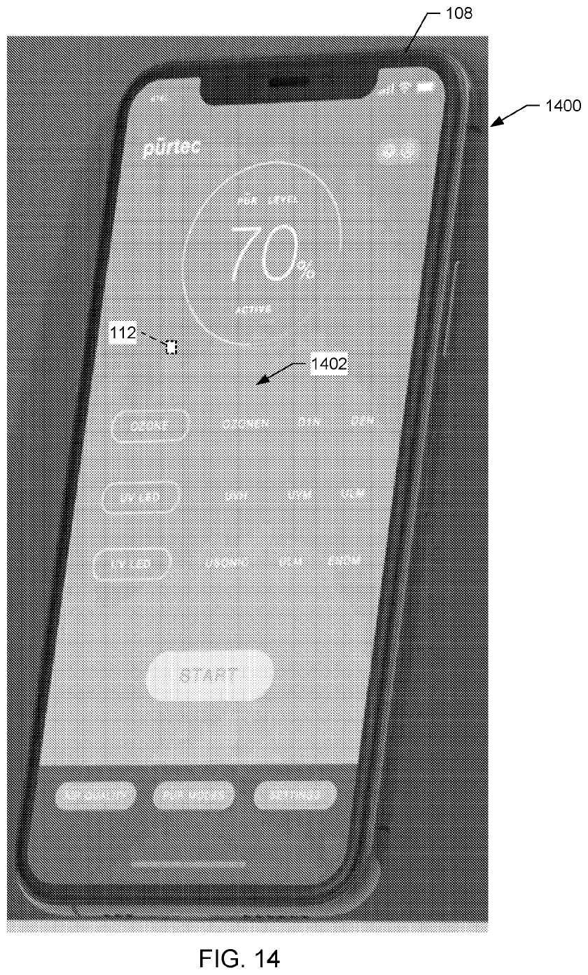

FIG. 13 is a diagram of a housing 1302 of the node 104, according to an example embodiment of the present disclosure. The housing 1302 is configured to have a disk shape, which provides a lower profile compared to the beacon apparatus 102. The node 104 is configured to attach to a wall, ceiling, etc. to provide sterilization and/or purification in small or hard to reach location. The display screen 222 may display a time when status or settings are not displayed, enabling the node 104 to be mounted as a clock.

As shown in FIG. 13, a front face of the housing 1302 includes at least three LEDs 230. The housing 1302 contains the active air generator 234 and the fan 236, which pulls in ambient air via a vent located in a back of the node 104. Air/ozone is dispersed via a front vent 1304. The node 104 also includes at least one proximity sensor 220 for detecting a presence of an individual. Operation of the node 104 in conjunction with the beacon apparatus 102 is discussed below in connection with the application 112.

### Application Embodiments

As discussed above, an application 112 is configured for use on a user device 108. The application 112 is in communication with one or more beacon apparatuses 102 and/or nodes 104. The application 112 is also in communication with the server 120. As discussed below, the application 112 is configured to provide control of the beacon apparatus 102 and/or node 104. Further, the application 112 is configured to display alerts, a status of the devices 102 and/or 104, and/or measured air quality and/or environmental conditions.

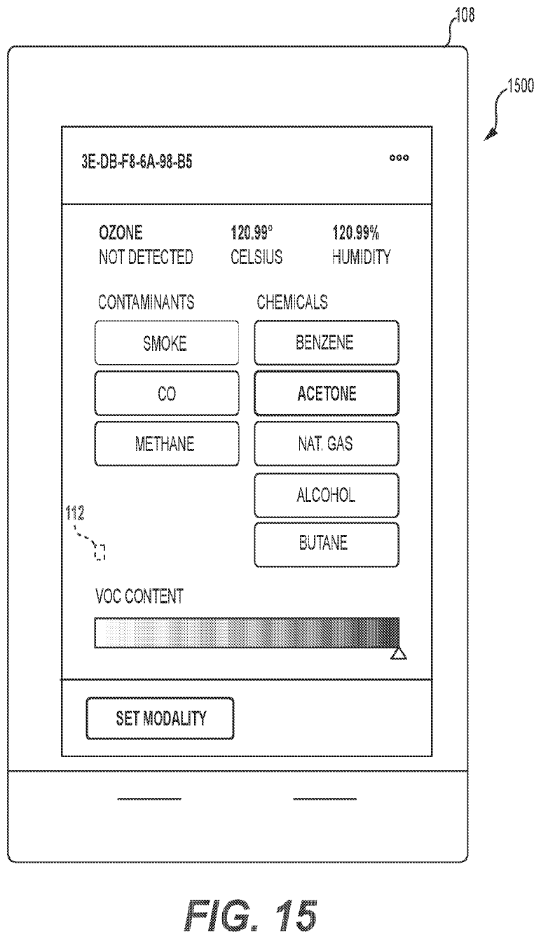

FIG. 14 is a diagram of a user interface 1400 of the application 112 displayed on the user device 108, according to an example embodiment of the present disclosure. The user interface 1400 displays a current air quality status, which is shown as a score of 70%. The application 112 may calculate the score taking into account a presence and/or concentration of certain detectable gases and/or containments. The user interface 1400 also displays a control section 1402 with different selectable options to control the beacon apparatus 102 and/or the node 104. The control section 1402 includes options for activating a purification/sterilization modality, such as the LEDs 230. The control section 1402 also lists different programs defined by the instructions 206 that operate a defined purification/sterilization routine. For example, selection of a ‘MN’ program causes the application 112 to send an instruction message to the beacon apparatus 102 to, for example, operate the active air generator for 60 minutes and the LEDs 230 for 80 minutes. The application 112 is configured to enable a user to program the different modalities and durations for each program.

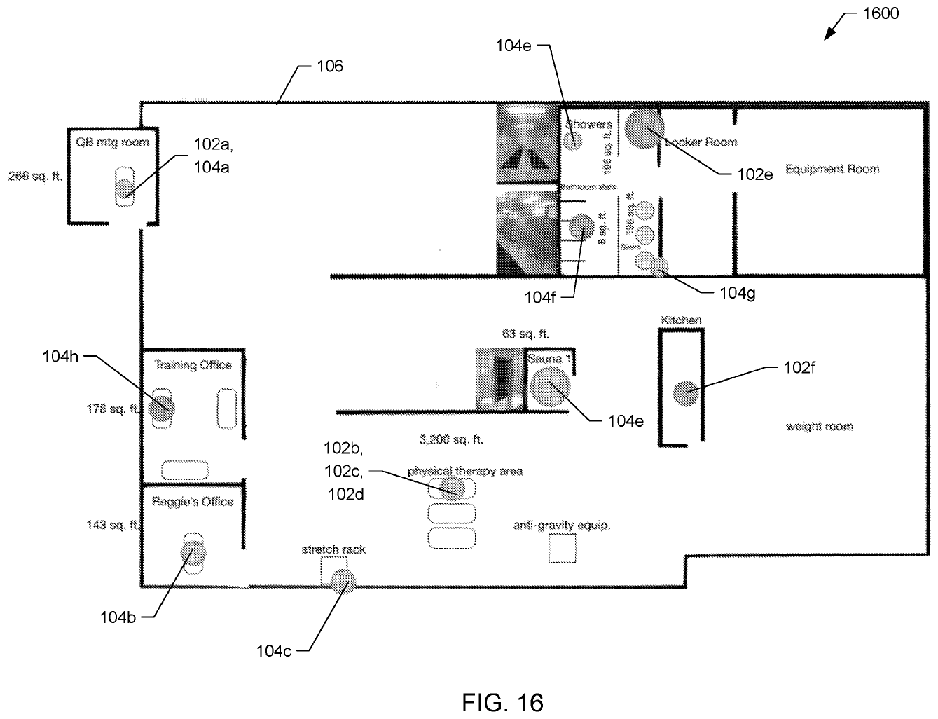

FIG. 15 is a diagram of a user interface 1500 of the application 112 displayed on the user device 108, according to an example embodiment of the present disclosure. The user interface 1500 is configured to display air quality information and/or environment information. For air quality, the application 112 displays ozone content, VOC content, and detected air containments including smoke, carbon monoxide, carbon dioxide, methane, benzene, acetone, natural gas, alcohol, and butane. The application 112 may color a box for each air containment based on a detected concentration. The user interface 1500 also includes environment conditions of the ambient air including temperature and relative humidity. The user interface 1500 includes an option ‘Set Modality’ to enable a user to define a program and/or conditions for activating air/surface purification and/or sterilization.

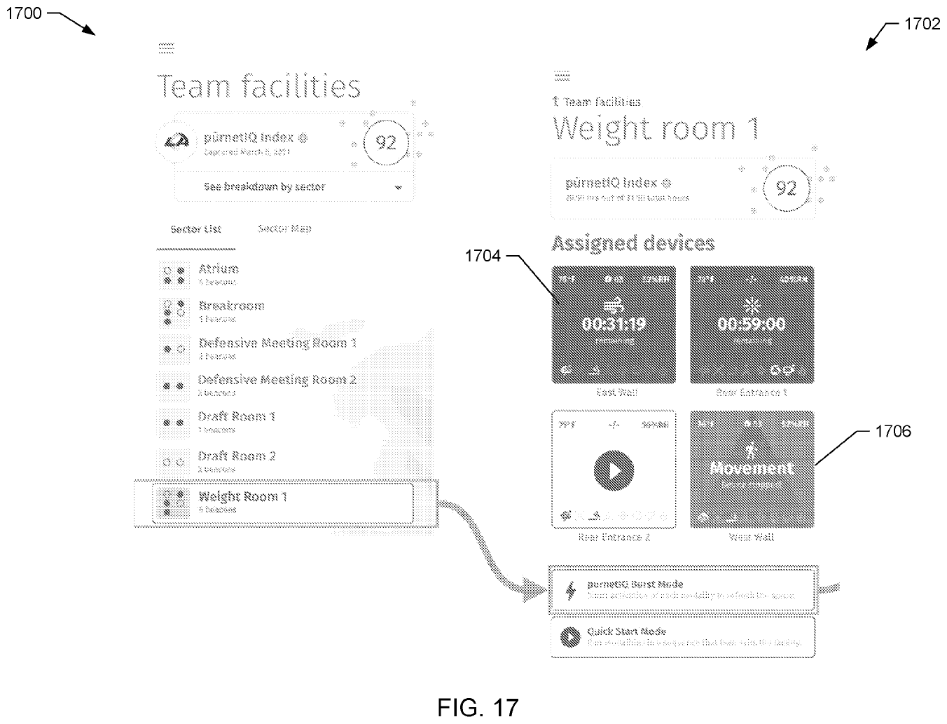

FIG. 16 is a diagram of a user interface 1600 showing beacon apparatuses 102 and nodes 104 in an indoor area 106, according to an example embodiment of the present disclosure. The user interface 1600 is displayed by the application 112 on the user device 108 to show an overall status of the indoor area 106 using air quality data from all beacons apparatuses 102 and nodes 104 registered to a common account.

The application 112 receives the air quality information from each beacon apparatus 102 and/or node 104, which is then aggregated and analyzed to determine an air quality per room. For example, the QB Meeting Room is assigned a beacon apparatus 102a and a node 104a. The application 112 receives air quality and/or status data from the beacon apparatus 102a and the node 104a, combines or averages the data, and determines an overall air quality or status for the room. The application 112 may receive the data directly from the devices 102a and 104a via a local network. Alternatively, the application 112 receives the data from the server 120, which receives the data from the beacon apparatus 102a and the node 104a.

In the illustrated example, areas shown in one color are indicative of a high level of purification while areas shown in another color are indicative of a lower level of purification. A user may select a room, causing the application 112 to display another user interface with air quality information specific for the room, such as the user interface 1500. Selection of a room also enables a user to transmit command messages to one or more of the devices 102 and/or 104 within that area.

To create the user interface 1600, the application 112 enables a user to upload a floor plan or create a floor plan. The application 112 also compiles a list of registered beacon apparatuses 102 and/or nodes 104. A user indicates which beacon apparatuses 102 and/or nodes 104 are located in each room or area. The indication may include dragging and dropping an icon of the beacon apparatus 102 and/or the node 104 to a location on the floor plan. The indication may also include assigning labels to each room or area of the floor plan, and assigning a corresponding label to the beacon apparatus 102 and/or the node 104.

As discussed above, the nodes 104 are installed to detect contaminants in local environments and on surfaces where standard sterilization methods are difficult to use or limited in coverage. The beacon apparatus 102 is used in larger spaces. As a combined system, the beacon apparatuses 102 and nodes 104 can be scaled into an unlimited number of spaces while communicating virtually over any network such as Wi-Fi or Bluetooth®. Each fan 236 of the beacon apparatus 102 and node 104 may be controlled by a respective processor 202 based on a size of a space the device 102 or 104 is located. For example, after detecting or receiving information that the beacon apparatus 102b is in a large physical therapy area of the indoor area 106 of FIG. 16, the processor 202 of that device causes the fan 236 to operate at a greater speed for optimal dispersion of ozone. Mobile network connectivity with the application 112 and the server 120 enables users to operate purification as a network platform rather than a single-room sterilization solution.

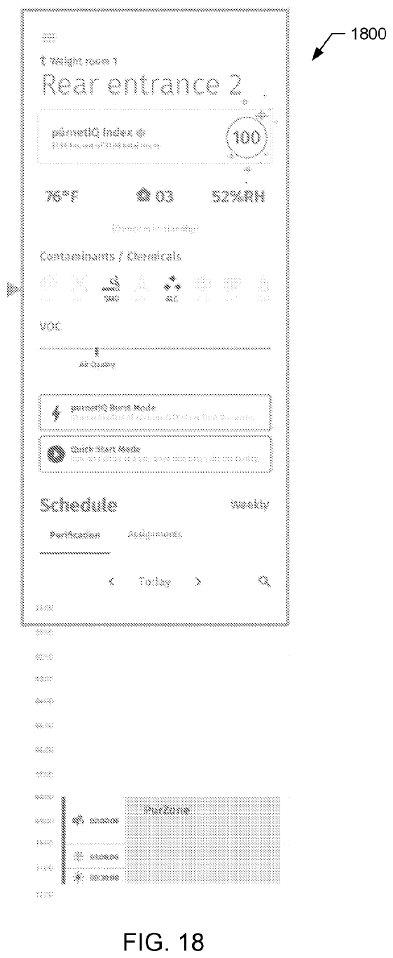

FIG. 17 shows a user interface 1700 providing a list of rooms in the indoor area 106 that have at least one beacon apparatus 102 and/or node 104, according to an example embodiment of the present disclosure. The user interface 1700 indicates for each room a number of beacon apparatuses 102 and/or nodes 104. The user interface 1700 also provides an indication as to whether each beacon apparatus 102 and/or node 102 is active.

The user interface 1700 further provides an index that is indicative of air quality. The index provides a custom space grade/score using data from one or more of the sensors 208 to 218 and device 102 and/or 104 run times to calculate relative conditions of a monitored indoor space. An overall area may have an index in addition to each room in the space having an index. The overall index may be an average or weighted average of indices of rooms/areas that comprise the overall space.

Selection of a room in the user interface 1700 causes the application 112 to display user interface 1702. The example user interface 1702 shows an operational status of each assigned device 102 and/or 104. For example, icon 1704 shows that the beacon apparatus 102 has 31 minutes and 19 second remaining for generating ozone. The icon 1704 also indicates that smoke and carbon monoxide have been detected. Another icon 1706 shows that the modalities have been paused because movement or a presence of an individual has been detected by the corresponding beacon apparatus 102.

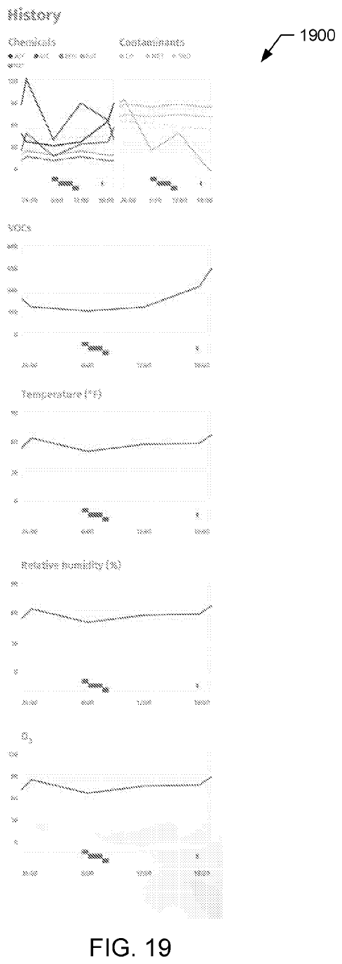

Section of one of the icons 1704, 1706 causes the application 112 to display user interface 1800 of FIG. 18. The user interface 1800 displays air quality metrics as detected by the corresponding beacon apparatus 102. In the illustrated example, the user interface 1800 shows that the beacon apparatus 102 has detected smoke and alcohol vapors. In some embodiments, the application 112 may display an alert notification if the concentration of smoke or alcohol vapors exceeds a threshold.

The user interface 1800 also shows that the VOC air quality is above average, the temperature is 76° F. and the relative humidity is 52%. The user interface 1800 also provides a device status of standby and options for a burst mode or quick start mode. The user interface 1800 also shows a schedule for when the beacon apparatus 102 is activate the purification/sterilization modalities, including an operational duration. The user interface 1800 enables a user to modify the schedule and/or modify conditions under when the beacon apparatus 102 is to activate. The user interface 1800 shows, for example, a view of scheduling capabilities for users to enact pre-set purification/sterilization times.

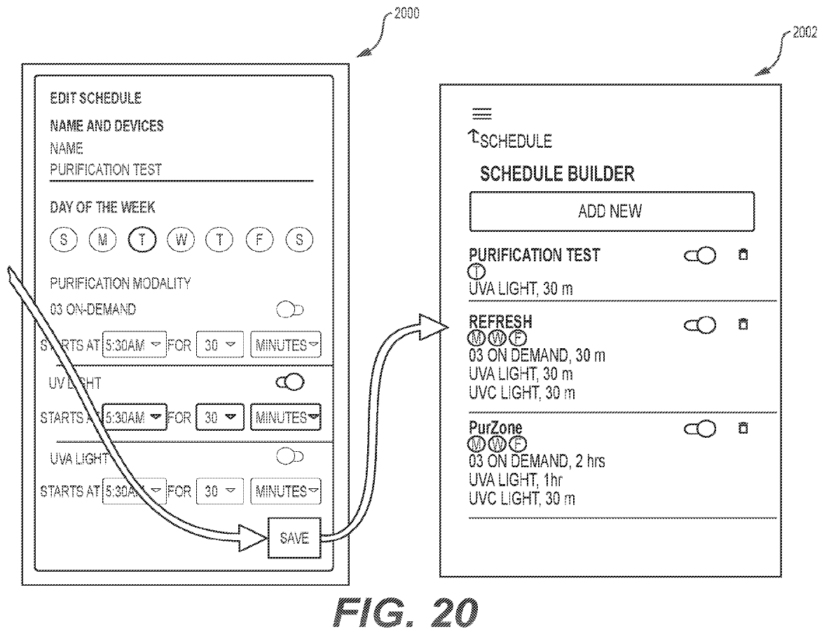

FIG. 19 is a diagram of a user interface 1900 showing an air quality history detected by the beacon apparatus 102, according to an example embodiment of the present disclosure. The history includes concentration trends of detectable chemicals and contaminants. The history also includes a history of VOCs, ozone, and environmental conditions including temperature and relative humidity. The application 112 may compile the data points shown in the user interface 1900. Additionally or alternatively, the application 112 may receive the trended history from the server 120, which may aggregate the data points.

In some embodiments, the application 112 and/or the server 102 may analyze the data to determine trends for providing recommendations. For example, the application 112 may use the data shown in the user interface 1900 to determine that VOCs tend to increase between 1200 and 20:00. In response, the application 112 (and/or the server 120) may provide a recommendation to start the active air generator 234 of the beacon apparatus 102 periodically between 12:00 and 20:00 to neutralize the VOCs. In some embodiments, during a purification cycle, the application 112 may monitor for spikes in contaminants and automatically adjust purification levels between the three modalities (e.g., UV-C and/or UV-A light, ozone, and ultrasonic sound waves).

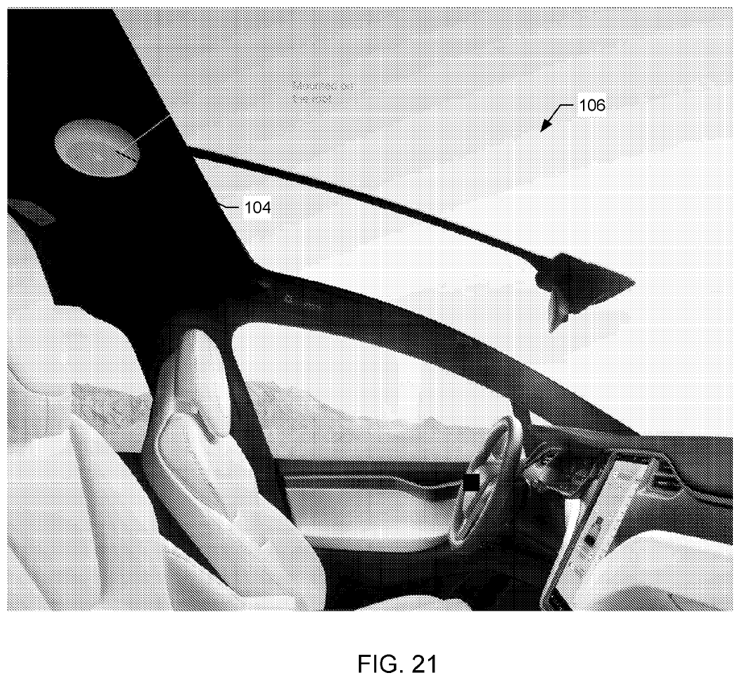

FIG. 20 is a diagram of a user interface 2000 of the application 112 that enables a user to schedule one or more modalities to activate on a beacon apparatus 102 or node 104, according to an example embodiment of the present disclosure. The user interface 2000 includes fields for a user to enter a program name, days of the week, and a start time for each purification/sterilization modality. The user interface 2000 also includes fields for a duration of each modality. It should be appreciated that the different modalities may be active at different types and for different durations. Further, not all three modalities need to be active for a given program. Other user interfaces 2000 enable a user to select conditions, such as air quality conditions, when one or more modalities are to be activated, as discussed above in connection with FIGS. 9 and 10.

After a time/date program/condition is created, the application 112 stores the program/condition to a schedule, as shown in the user interface 2002. The schedule identifies different programs/conditions under which one or more beacon apparatuses 102 and/or nodes 104 are to provide indoor decontamination and/or purification. The application 112 is configured to transmit the programs/conditions to the corresponding beacon apparatus 102 and/or node 104, which is stored as the instructions 206 in the memory device 204.

In alternative embodiments, the application 112 and/or the server 120 may store the programs/conditions. In these alternative embodiments, the application 112 and/or server 120 only transmits activation instructions to the beacon apparatus 102 and/or the node 104 indicating which modalities are to be activated. At a scheduled deactivation, the application 112 and/or the server 120 transmits an instruction to the beacon apparatus 102 and/or the node 104 indicating which of the modalities are to be deactivated.

Use Case Embodiments

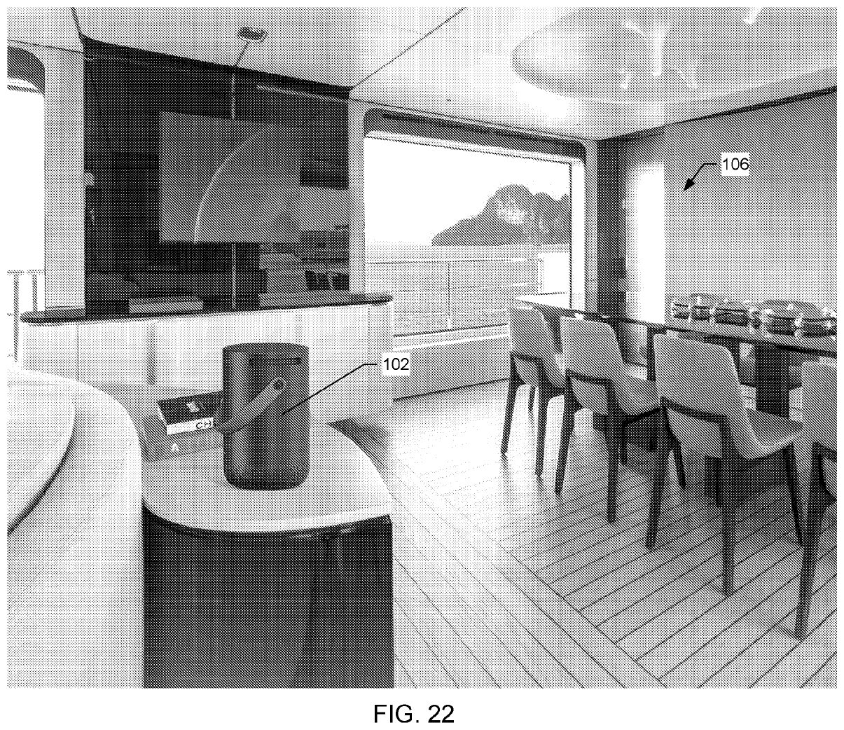

The above embodiments showed the beacon apparatus 102 and/or the node 104 deployed in a building or residence. The beacon apparatus 102 and/or the node 104 may be deployed in other environments and/or structures. For example, FIG. 21 is a diagram of the node 104 installed in a car. FIG. 22 is a diagram of the beacon apparatus 102 installed in a cabin of a cruise boat. In other embodiments, the beacon apparatus 102 and/or the node 104 may be installed in an airplane, train, subway, rideshare vehicle, etc. Further, the beacon apparatus 102 and/or the node 104 may be installed within a hotel. The interconnectivity of the devices 102 and 104 enable an operator to manage them collectively across a facility. Alternatively, an operator may enable a guest to connect and control the device 102/104 that is assigned to room/space. In yet other embodiments, the nodes 104 maybe configured as smaller wearable devices for personal use. The nodes 104 may be attachable to clothing or may be carried in a purse.

### Alternative Embodiments

In some embodiments, the beacon apparatus 102, nodes 104, and/or the application 112 are configured to communicate with pre-existing network-enabled HVAC systems to provide additional indoor environment control. Further, the beacon apparatus 102 and/or the nodes 104 can include hydroxyl generators for an additional purification modality. The beacon apparatus 102 and/or the nodes 104 may also include a photocatalytic filter as another modality.

Further, in some embodiments, the beacon apparatus 102 may be mounted on or integrated within a robotic cart. Beacon apparatus 102 disclosed herein connectivity enables a user to remotely control the cart and/or specify a path of travel. In other instances, the beacon apparatus 102 may use machine learning and/or artificial intelligence navigation to circumvent an indoor area to increase a range of purification/sterilization.

### CONCLUSION

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A purification and sterilization apparatus comprising:
a housing including a top side, a bottom side, a cylindrical face between the top side and the bottom side;
at least one ultraviolet lighting emitting diode (“LED”) supported by the housing;
an active oxygen generator located within the housing;
at least one proximity sensor located within the housing;
a processor located within the housing; and
a memory storing machine readable instructions, which when executed by the processor, cause the processor to:
activate at least one of the active oxygen generator or the at least one ultraviolet LED to provide air/surface purification and/or sterilization at a designated time, a designated condition, or upon receiving an instruction,
receive a signal from the at least one proximity sensor indicative of a presence individual,
pause activation of at least one of the active oxygen generator or the at least one ultraviolet LED, and
resume activation of at least one of the active oxygen generator or the at least one ultraviolet LED when the presence of the individual is no longer detected for at least a time threshold.

2. The apparatus of claim 1, further comprising at least one air sensor located within the housing, and wherein the memory stores additional machine readable instructions, which when executed by the processor, cause the processor to:
determine activation of the active oxygen generator is to be stopped based on the designated time or upon receiving a second instruction;
determine from at least one signal from the air sensor that an ozone concentration is above an ozone threshold; and
cause the at least one ultraviolet LED to activate to reduce the ozone concentration below the ozone threshold.

3. The apparatus of claim 2, wherein the ozone threshold is at least 20 parts per billion.

4. The apparatus of claim 2, wherein the at least one air sensor includes at least one of a formaldehyde sensor, one or more air component sensors, or one or more volatile organic compound (“VOC”) sensors, and
the designated condition includes a detection by the processor of a containment above a threshold level using at least one signal from the at least one air sensor.

5. The apparatus of claim 4, wherein the one or more air component sensors are configured to provide for the detection of at least one of ozone, carbon dioxide, combustible gas/smoke, alcohol vapors, methane, propane, butane, liquefied petroleum, liquid natural gas, carbon monoxide, hydrogen, ozone, ammonia sulfide, or benzene vapor.

6. The apparatus of claim 2, wherein the at least one air sensor includes at least one of a temperature sensor, a humidity sensor, or a barometric pressure sensor.

7. The apparatus of claim 6, wherein the memory stores additional machine readable instructions, which when executed by the processor, cause the processor to:
determine a relative humidity of ambient air is greater than a humidity threshold; and
activate the active oxygen generator while refraining from activing the at least one ultraviolet LED.

8. The apparatus of claim 7, wherein the memory stores additional machine readable instructions, which when executed by the processor, cause the processor to:
determine the relative humidity of ambient air is less than the humidity threshold; and
activate the at least one ultraviolet LED while refraining from activing the active oxygen generator.

9. The apparatus of claim 8, wherein the humidity threshold is between 65% and 75% relative humidity.

10. The apparatus of claim 1, wherein the top side of the housing includes a cylindrical section, and
wherein a plurality of the ultraviolet LEDs are placed around a circumference of the cylindrical section.

11. The apparatus of claim 10, further comprising:
a motor; and
an actuator arm connected to the motor and the cylindrical section,
wherein the motor is configured to cause the actuator arm to raise and lower the cylindrical section with respect to the housing such that the plurality of the ultraviolet LEDs are exposed when the cylindrical section is in a raised position and hidden from view when the cylindrical section is in a retracted position.

12. The apparatus of claim 10, wherein at least some of the plurality of the ultraviolet LEDs are configured to emit light in the 250 to 270 nanometer (“nm”) wavelength range and other of the at least some of the plurality of the ultraviolet LEDs are configured to emit light in the 390 to 420 nm wavelength range.

13. The apparatus of claim 1, wherein the cylindrical face includes:
a first vent adjacent to the top side; and
a second vent adjacent to the bottom side.

14. The apparatus of claim 13, wherein the active oxygen generator includes:
an ozone ionizer plate; and
a fan configured to pull ambient air through the second vent and cause ozone to be emitted through the first vent when the ozone ionizer plate is active.

15. The apparatus of claim 1, wherein the time threshold is between five seconds and fifteen minutes.

16. The apparatus of claim 1, further comprising at least one ultrasonic speaker within the housing, the at least one ultrasonic speaker configured to emit a waveform having a frequency between 20 and 80 kHz, a sound pressure level between 80 and 150 dB, and an angle of radiation between 45° and 180°.

17. The apparatus of claim 1, further comprising a display screen provided on the cylindrical face and including at least one of a touchscreen or input buttons,
wherein the memory stores additional machine readable instructions, which when executed by the processor, cause the processor to receive the instruction via the display screen.

18. The apparatus of claim 17, wherein the memory stores additional machine readable instructions, which when executed by the processor, cause the processor to display a status or an air quality indication on the display screen.

19. The apparatus of claim 1, further comprising a transceiver for communicatively coupling the processor to a user device,
wherein the memory stores additional machine readable instructions, which when executed by the processor, cause the processor to receive the instruction via the transceiver from an application operating on the user device.

20. The apparatus of claim 19, wherein the memory stores additional machine readable instructions, which when executed by the processor, cause the processor to transmit a status or an air quality indication for display by the application on the user device.

21. The apparatus of claim 1, further comprising a transceiver for communicatively coupling the processor to another purification and sterilization apparatus or a hub configured as a smaller version of the purification and sterilization apparatus.
